(19) United States
(12) Patent Application Publication
(10) Pub. No.: US 2026/0007724 A1
(43) Pub. Date: Jan. 8, 2026

(54) METHODS AND COMPOSITIONS FOR IMPROVING WOUND HEALING

(71) Applicant: The United States of America, as represented by the Secretary, Dept. of Health and Human Services, Bethesda, MD (US)

(72) Inventors: Kaitlyn Noelle Sadtler, Bethesda, MD (US); Ravi Lokwani, Bethesda, MD (US); Tran Ngo, Bethesda, MD (US)

(21) Appl. No.: 18/992,393

(22) PCT Filed: Jul. 7, 2023
(86) PCT No.: PCT/US2023/069798

§ 371 Date: Jan. 8, 2025

Related U.S. Application Data
(60) Provisional application No. 63/367,994, filed on Jul. 8, 2022.
(60) Provisional application No. 63/488,122, filed on Mar. 2, 2023.

(51) Int. Cl. A61K 38/39; A61K 31/728; A61K 31/737; A61K 35/12; A61K 38/01; A61P 17/02; A61P 37/02

(52) U.S. Cl. CPC A61K 38/39; A61K 31/728; A61K 31/737; A61K 35/12; A61K 38/014; A61P 17/02; A61P 37/02

(57) ABSTRACT

The present disclosure relates to compositions and methods for improving wound healing and tissue regeneration. More specifically, the present disclosure relates to compositions, and methods of using such compositions, that direct the immune response within a wound towards a pro-regenerative response. Such compositions and methods are particularly useful in altering the immune response elicited by medical implants, to avoid scarring and fibrosis at the site of implantation.

### PRIORITY PARAGRAPH

This application claims the benefit of priority under 35 U.S.C. § 119 of U.S. Provisional Application Ser. No. 63/367,994 filed on Jul. 8, 2022, U.S. Provisional Application Ser. No. 63/488,122 filed on Mar. 2, 2023, the contents of which are incorporated herein by reference in their entirety.

### STATEMENT REGARDING FEDERALLY SPONSORED RESEARCH & DEVELOPMENT

This work was supported by the Division of Intramural Research at the National Institute of Biomedical Imaging and Bioengineering, an institute within the National Institutes of Health. The U.S. government has certain rights in this invention.

### BACKGROUND

The field of the disclosure generally relates to wound healing and tissue engineering. The goal of tissue engineering is to replace the function of missing or damaged tissues and organs. This may be accomplished using biomaterial scaffolds that integrate and help regenerate injured or missing tissue such as skin grafts, as well as medical device implants made of synthetic materials to replace the function or cosmesis of that tissue such as knee replacements (see, for example, US2019/0060524, which is incorporated herein by reference in its entirety). However, any time a biomaterial or medical device is implanted in the human body, it alters homeostasis and induces a cascade of immune responses that can either positively lead to scaffold integration and tissue growth or yield immune mediated pathologies such as implant fibrosis or excessive inflammation and surrounding tissue damage. As such, understanding the mechanism by which our immune system interacts with engineered materials is necessary to produce next-generation medical devices capable of working with the immune system.

The field of evaluating immune responses to engineered materials has grown over the past decade and resulted in numerous findings regarding how immune cells may moderate material acceptance or rejection and fibrosis. Integrating with fields of wound healing and developmental biology several cells have been implicated in these processes. Specifically, type 2 immunity including eosinophils, M2 macrophages, and Th2 T cells along with the signaling protein interleukin (IL)-4 have been described in healing a variety of tissues including liver, nerve, skin, and muscle. Regulatory T cells and associated cytokines (IL-10) have also been described in healing processes and have been utilized in material acceptance strategies to try to inhibit excessive immune activation. In terms of pathology, Th17 T cells that have been implicated in tissue fibrosis and autoimmunity, have also been linked to device fibrosis in mice and humans. Crosstalk between these immune cells such as T cells, macrophages, along with fibroblasts, generates an intricate network of cell signaling wherein multiple factors contribute to regeneration and acceptance or inflammation and fibrosis.

While research to date has begun to illuminate the cellular processes involved in wound healing and fibrosis, treatment success using existing methods remains inconsistent, the treatment of wounds and avoidance of fibrosis remains a challenge. Thus, there is a need for better and more specific treatments that allow regeneration of tissue within wounds without accompanying fibrosis. The present disclosure addresses this need.

### SUMMARY

One aspect is a method comprising administering at the site of a wound a therapeutic composition that induces a pro-regenerative environment within the wound and/or within tissue surrounding the wound.

One aspect is a method of altering an immune response to a wound, comprising administering at the site of the wound a therapeutic composition that induces a pro-regenerative environment within the wound and/or within tissue surrounding the wound.

One aspect is a method of treating a wound in an individual, comprising administering at the site of the wound a therapeutic composition that induces a pro-regenerative environment within the wound and/or within tissue surrounding the wound.

One aspect is a method of treating an individual having a wound, comprising administering at the site of the wound a therapeutic composition that induces a pro-regenerative environment within the wound and/or within tissue surrounding the wound.

One aspect is a method of reducing or preventing fibrosis in a wound, comprising administering at the site of the wound a therapeutic composition that induces a pro-regenerative environment within the wound and/or within tissue surrounding the wound.

One aspect is a method of implanting a medical device in an individual, comprising introducing the medical device into tissue within the individual, and administering at the site of the implanted medical device a therapeutic composition that induces a pro-regenerative environment within the wound and/or within tissue surrounding the wound.

One aspect is a method of implanting a medical device in an individual, comprising introducing the medical device into tissue within the individual, wherein a therapeutic composition that induces a pro-regenerative environment within the wound and/or within tissue surrounding the wound is applied to the medical device prior to its implanting in an individual.

In these aspects, the wound may be a burn, a contusion, a seroma, a hematoma, a laceration, an avulsion, a puncture, a surgical wound, an incision, an ulcer or a wound due to a crushing injury. In these aspects, administering the therapeutic composition may comprise introducing the therapeutic composition into the wound cavity and/or into tissue surrounding the wound cavity, which may comprise applying (e.g., topically) the therapeutic composition to skin surrounding the wound cavity.

In these aspects, the therapeutic composition may comprise an ointment, a spray, a lotion, a gel, a cream, a foam, a solution, a suspension, an emulsion, a hydrogel, or a paste, which may comprise nanoparticles or microspheres. In these aspects, the therapeutic composition may be formulated as a rapid-release composition or as a slow-release composition. In these aspects, the therapeutic composition may release the therapeutic agent over approximately 5 minutes, over approximately 10 minutes, over approximately 30 minutes, over approximately one hour, over approximately two hours, over approximately 12 hours, over approximately 24 hours, over approximately 48 hours, over approximately 5 days, over approximately one week, or over approximately one month or longer.

In these aspects, the therapeutic composition may comprise a therapeutic agent that induces a pro-regenerative environment. The therapeutic agent may direct the immune response away from a TH1-type response. The therapeutic agent may induce a TH2-driven immune environment. The therapeutic agent may increase the number of M2 macrophages in the wound and/or within the tissue surrounding the wound. The therapeutic agent may induce an influx of M2 macrophages within the wound and/or within the tissue surrounding the wound, and/or it may induce local proliferation of M2 macrophages within the wound and/or within the tissue surrounding the wound. The therapeutic agent may induce an increase in the number of conventional dendritic cells (cDC1s) within the wound and/or within the tissue surrounding the wound. The therapeutic agent may induce an influx of conventional dendritic cells (cDC1s) within the wound and/or within the tissue surrounding the wound, and/or it may induce local proliferation of cDC1s within the wound and/or within the tissue surrounding the wound. The cDC1s may be cross-presenting dendritic cells. The cDC1s may be XCR1⁺CD103⁺dendritic cells.

In these aspects, the therapeutic agent may comprise a peptide, a protein, a glycoprotein, a lipoprotein, a lipid, a sugar, a polysaccharide, a glycosaminoglycan, a nucleic acid molecule, an organic molecule, and combinations thereof. In these aspects, the therapeutic agent may comprise decellularized extracellular matrix (ECM), or a component derived therefrom, which may comprise a degradation product of ECM. In these aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin, and degradation products thereof. The therapeutic agent may comprise a matrikine, which may be selected from the group consisting of metastatin, affesten, canstatin, tetrastatin, pentastatin, lamstatin, hexastatin, endotrophin, restin1, restin2, restin3, restin4, endostatin, neostatin, anastellin, sibsttin, PEX, endorepellin, CUBICUB2 domain, Ten/2, Ten11/12/13, Ten14, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin and any combination thereof. In certain aspects, the therapeutic agent may comprise a DAMP.

In certain aspects, the medical device may be an implant, which may be selected from the group consisting of a breast implants, a stent, a port, a shunt, a hip implant, a knee implant, a cochlear implant, hernia, or other, surgical mesh, an intraocular lens implant, a pacemaker, a metal/surgical screw, rod, or pin, an artificial disc, and spinal fusion hardware. In certain aspects, the implant may comprise metal and metal alloys, plastic polymers, ceramics, hydrogels and composites, which may include, but which are not limited to, silicone, polyethylene, stainless steel, titanium, zirconia, polyurethane foam, polylactic acid, amalgam, gold, alumina, silicate, chrome, cobalt, and molybdenum.

One aspect is a therapeutic composition for treating a wound, wherein the therapeutic composition comprises a therapeutic agent that induces a pro-regenerative environment within the wound and/or within the tissue surrounding the wound. The therapeutic composition may comprise an ointment, a spray, a lotion, a gel, a cream, a foam, a solution, a suspension, an emulsion, a hydrogel, or a paste, which may comprise nanoparticles or microspheres. The therapeutic composition may be formulated as a rapid-release composition, or as a slow-release composition. The therapeutic composition may release the therapeutic agent over approximately 5 minutes, over approximately 10 minutes, over approximately 30 minutes, over approximately one hour, over approximately two hours, over approximately 12 hours, over approximately 24 hours, over approximately 48 hours, over approximately 5 days, over approximately one week, or over approximately one month or longer. In certain aspects, the therapeutic composition may comprise a therapeutic agent that induces a pro-regenerative environment. The therapeutic agent may direct the immune response away from a TH1-type response. The therapeutic agent may induce a TH2-driven immune environment. The therapeutic agent may increase the number of M2 macrophages in the wound and/or within the tissue surrounding the wound. The therapeutic agent may induce an influx of M2 macrophages within the wound and/or within the tissue surrounding the wound, and/or it may induce local proliferation of M2 macrophages within the wound and/or within the tissue surrounding the wound. The therapeutic agent may induce an increase in the number of conventional dendritic cells (cDC1s) within the wound and/or within the tissue surrounding the wound. The therapeutic agent may induce an influx of conventional dendritic cells (cDC1s) within the wound and/or within the tissue surrounding the wound, and/or it may induce local proliferation of cDC1s within the wound and/or within the tissue surrounding the wound. The cDC1s may be cross-presenting dendritic cells. The cDC1s may be XCR1⁺CD103⁺dendritic cells. The therapeutic agent may comprise a peptide, a protein, a glycoprotein, a lipoprotein, a lipid, a sugar, a polysaccharide, a glycosaminoglycan, a nucleic acid molecule, an organic molecule, and combinations thereof. In certain aspects, the therapeutic agent may comprise decellularized extracellular matrix (ECM), or a component derived therefrom, which may comprise a degradation product of ECM. The therapeutic agent may comprise one or more components selected from the group consisting of collagen, laminin, fibronectin, elastin, chondroitin sulfate, heparan sulfate, keratan sulfate, hyaluronic acid, perlecan, agrin, and degradation products thereof. The therapeutic agent may comprise a matrikine, which may be selected from the group consisting of metastatin, affesten, canstatin, tetrastatin, pentastatin, lamstatin, hexastatin, endotrophin, restin1, restin2, restin3, restin4, endostatin, neostatin, anastellin, sibsttin, PEX, endorepellin, CUBICUB2 domain, Ten/2, Ten11/12/13, Ten14, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a vVGVAPG peptide, a PGP tripeptide, an acetylated PGP tripeptide (AcPGP), tenascin-C (TNC), the G3 domain of nidogen-1, tumstatin, and any combination thereof. The therapeutic agent may comprise a DAMP.

One aspect of the disclosure is a method of recruiting NK cells to a wound, or to tissue proximal to a wound, comprising administering at the site of wound a therapeutic agent or a therapeutic composition of the disclosure.

One aspect of the disclosure is a method of activating an NK cell, comprising contacting the NK cell with therapeutic agent of the disclosure.

One aspect of the disclosure is a method of inducing increased expression of Xcl1 in an NK cell, comprising contacting the NK cell with therapeutic agent of the disclosure.

One aspect of the disclosure is a kit comprising a therapeutic composition of the disclosure. The kit may also comprise needles, syringes, vials, applicators, and instructions for using the therapeutic composition for treating a wound.

One aspect of the disclosure is the therapeutic composition of the disclosure when used for treating wound, wherein the therapeutic composition induces a pro-regenerative environment in the wound and/or within tissue surrounding the wound.

One aspect of the disclosure is the therapeutic composition of the disclosure when used for altering an immune response to a wound, wherein the therapeutic composition induces a pro-regenerative environment in the wound and/or within tissue surrounding the wound.

One aspect of the disclosure is the therapeutic composition of the disclosure when used for reducing or preventing fibrosis in a wound, wherein the therapeutic composition induces a pro-regenerative environment in the wound and/or within tissue surrounding the wound.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows the gating strategy for a myeloid panel. Representative plots and gates from sample stained with 22 color myeloid phenotyping panel. Example data are from 7 days post-injury.

FIGS. 2A-C show immune cell infiltration into muscle injury. Cells were counted on a hemocytometer prior to flow staining, live immune cell counts are displayed. FIG. 2A shows individual values per mouse; FIG. 2B shows mean±SEM. FIG. 2C shows hematoxylin and eosin staining of muscle injury at 7 days post-injury.

FIGS. 3A-D show pro-regenerative and pro-fibrotic materials recruit a diverse range of innate immune cells. Innate immune cell prevalence in biomaterial treated muscle injury. FIG. 3A: t-stochastic neighbor embedding (t-SNE) (left); Uniform Manifold Approximation and Projection (UMAP) (middle); and FlowSOM (right) dimensionality reduction calculations displayed against manually gated populations at 7 days post-injury. Macrophages=red, Dendritic Cells=blue, Monocytes=green, Eosinophils=light blue, Basophils=black, Neutrophils=orange, Other immune cells=purple. FIG. 3B: t-SNE (left) UMAP (middle) and FlowSOM (right) dimensionality reduction calculations displayed against injury treatment type. Control=black, ECM-tx=teal, PE-tx=pink. FIG. 3C: t-SNE (left) UMAP (middle) and FlowSOM (right) dimensionality reduction calculations displayed against computationally derived clusters (FlowSOM). FIG. 3D shows phenotyping of computationally derived clusters. Expression=scaled fluorescence intensity.

FIGS. 4A-E show immune cell populations over time as a percent of live CD45+ immune cells. Basophils: CD11b-CD200R3+, Eosinophils: CD11b+Siglec-F+, Neutrophils: CD11b+Ly6G+, Total Monocytes (CD11b+Ly6C+), Classical Monocytes Ly6Chi, CX3CR1lo, Alternative Monocytes: Ly6C+CX3CR1hi, Non-DC antigen presenting cells (CD11b-CD11c-MHCII+), Dendritic Cells: CD11b-CD11c+MHCII+, Total macrophages: CD11b+CD68 and or F4/80+, Macrophages: F4/80+, Macrophages F4/80⁺CD68+, Macrophages: CD68+. Control=black, ECM treated=teal, PE treated=pink. Data are means±95% Confidence Interval, n=5.

FIGS. 5A & 5B show phenotyping of MHCII+ antigen presenting cells in the wound microenvironment at 7 days post injury. FIG. 5A shows percent of MHCII+ cells that are identified as listed immune cell types. FIG. 5B shows proportion of each treatment group that is represented by different cell types. SD=standard deviation. Black=neutrophils, pink=eosinophils, teal=macrophages, purple=basophils, lavender=dendritic cells, blue=monocytes, grey=other immune cells. ANOVA with Tukey Post-hoc correction for multiple comparisons, *=P<0.05, **=P<0.01, ***=P<0.001, ****=P<0.0001.

FIGS. 6A-6C show phenotyping markers of macrophages and dendritic cells. FIG. 6A-MHCII+ macrophages (F4/80⁺CD68+MHCII+). FIG. 6B-MHCII− Macrophages (F4/80⁺CD68+MHCII−). FIG. 6C-Dendritic cells (CD11c+MHCII+). Black/grey=control injury; teal=ECM-treated injury; pink=PE-treated injury. n=5 data are shown as individual points with bar at mean.

FIGS. 7A-7H show that cross-presenting dendritic cells are enriched by pro-regenerative scaffolds and peak by 7 days post injury. FIG. 7A shows representative dot plots of CD45⁺CD11blo/−CD11c+MHCII+ dendritic cells at 7 days post-injury. Four quadrants correspond to: Q1=CD103−XCR1+, Q2=CD103+XCR1+, Q3=CD103+XCR1−, and Q4=CD103−XCR1−. FIG. 7B shows the proportion (%) of dendritic cells within four quadrants at 3-, 7-, 21-, and 42-days post-injury. FIG. 7C shows the count (in 10,000s) of dendritic cells per quad at 3-, 7-, 21-, and 42-days post-injury. FIG. 7D shows tSNE and FIG. 7E shows UMAP of CD45+ immune cells at 7-days post-injury concatenated from three treatment groups, cDC1 population highlighted in red. FIG. 7F shows the proportion of cDC1 population from three treatment groups. FIG. 7G shows phenotyping of 30 different immune cell clusters identified by FlowSOM algorithm, normalized within each marker where min=0, and max=100. FIG. 7H FlowSOM scaled expression values on different clusters with cluster 22 (cDCls) highlighted in red. Control=black, ECM treated=teal, PE treated=pink. CD103+XCR1+cDCs=red. Data are means±95% Confidence Interval, n=5 representative of at least two independent experiments.

FIGS. 8A-8D show dendritic cell subsets at (FIG. 8A) 3 days post-injury (FIG. 8B) 7 days post-injury, (FIG. 8C) 21 days post injury, and (FIG. 8D) 42 days post-injury. ANOVA with Tukey Post-hoc correction for multiple comparisons, *=P<0.05, **=P<0.01, ***=P<0.001, ****=P<0.0001. Data are means±SEM, n=5 representative of at least two independent experiments.

FIGS. 9A-9C show CD103 and XCR1 expression on myeloid cells. FIG. 9A shows CD103 mean fluorescence intensity, while FIG. 9B shows XCR1 mean fluorescence intensity on dendritic cells (black), MHCII+ macrophages (blue), and MHCII-macrophages (yellow). FIG. 9C shows F4/80− and F4/80+ fractions of XCR1+ cells purified (via MACS column) from an ECM-treated VML at 7 days post-injury. Red=actin/phalloidin, Blue=DAPI. N=4-5, ANOVA with Tukey post-hoc correction for multiple comparisons, *=P<0.05, **=P<0.01, ****=P<0.0001.

FIG. 10 shows CD86 expression on cDC1s. Representative FACS plots of CD11c+MHCII+ dendritic cells in three treatment groups. Green=XCR1+CD103+ cDCls; purple=XCR1−CD103− dendritic cells.

FIGS. 11A & 11B show repeatability of findings across litters, backgrounds and species. XCR1+CD103+ cells are present in multiple runs with mice from (FIG. 11A) different litters and (FIG. 11B) species. Student's T-test with Tukey post-hoc correction, ns=not significant. Rat data are extracted from Adusei et al, Cells, Tissues, and Organs, 2022.

FIG. 12 shows CD103+XCR1+ Dendritic cells in the skin overlying a muscle injury. At 7 days post-injury in an ECM-treated mouse, as a proportion of CD11c+MHCII+ dendritic cells. Data are means±standard deviation, n=3-5. Student's t-test, p=0.0003.

FIGS. 13A & 13B show Dendritic cell phenotype in local tissue, blood, and draining lymph node. FIG. 13A shows representative FACS plots, color scale=CD8a expression. FIG. 13B: purple=local muscle tissue, green=peripheral blood, blue=inguinal lymph node (LN).

FIGS. 14A-14C show peripheral plasmacytoid DCs are enriched with pro-fibrotic material treatment of local injury. (a) Innate-like cells in draining lymph node. (b) innate like cells in blood. (c) B220/CD45R expression on dendritic cells in the blood and LN. *=P<0.05; **=P<0.01, =P<0.001. n=5, ANOVA with Tukey post-hoc.

FIG. 15 shows XCL-1 levels in peripheral blood at 7- and 21-days post-injury. Y-axis=pg/ml in plasma, X-axis=weeks post-injury. Black=control, Teal=ECM-tx, Pink=PE-tx. Data are means±SEM, n=5. Student's T-test, **=P<0.01. Differences between treatment groups are not significant.

FIGS. 16A-16J show that Type-2 myeloid markers are enhanced on cross-presenting dendritic cells by pro-regenerative scaffold treatment and dependent upon adaptive immunity. FIG. 16A shows CD11c prevalence in muscle tissue as a proportion of CD45+ live immune cells. FIG. 16B shows CD103+XCR1+ dendritic cells. FIG. 16C shows CD103+/−XCR1+ dendritic cells. FIG. 16D shows CD103+XCR1+/− dendritic cells. FIG. 16E shows gating strategy for dendritic cells FIG. 16F shows representative FACS plots. Blue=Wild Type C57BL/6J mice, Red=Rag1−/− B6 mice. FIG. 16G shows CD301b expression on F4/80+ macrophages in wild type (WT) and RAG-deficient mice (Rag1−/−). FIG. 16H shows CD301b expression on cDC1's in WT and Rag1−/− mice. FIG. 16I shows CD206 expression on F4/80+ macrophages In WT and Rag1−/− mice. FIG. 16J shows CD206 expression on cDC1's in WT and Rag1−/− mice. ANOVA with Tukey Post-hoc correction for multiple comparisons, *=P<0.05, **=P<0.01, ***=P<0.001, ****=P<0.0001, n=5.

FIG. 17 shows a lymphoid panel gating strategy. Displayed is a representative sample from the inguinal lymph node at 21 days post-injury.

FIGS. 18A-18F show activation of T cells along with prevalence of HELIOS+ iTregs is temporally regulated and difference between draining lymph nodes and peripheral blood. FIGS. 18A-18B show activated T cells in the draining (inguinal) lymph node (ILN) and peripheral blood at 7 days (FIG. 18A) and 21 days post-injury (FIG. 18B). FIGS. 18C-18F show the proportion of FoxP3+ and HELIOS+ iTregs at 7 (FIGS. 18C-18D) days post injury and 21 days post injury (FIGS. 18E-18F) in the (18C & 18E) ILN and (18D & 18F) peripheral blood. ANOVA with Tukey Post-hoc correction for multiple comparisons, * P<0.05, **=P<0.01, ***=P<0.001, ****=P<0.0001. n=5.

FIG. 19 shows HELIOS+ regulatory CD8s over time. Dark grey=7 days post-injury. Light grey=21 days post-injury. ANOVA with Kruskal-Wallis FDR post-hoc.

FIGS. 20A & 20B show ST2 expressing B-cell and pDCs in the lymph node after injury.

FIGS. 21A-21E show that trauma induces proliferation and activation of CD103+XCR1+ adaptive immune cells in the draining lymph node.

FIG. 21A is a representative dot plot from draining (inguinal) lymph node from an uninjured mouse and those from the DLN of an injured mouse at 21 days post-injury. B cells=CD45+CD11c-CD11b-CD3-B220+, T cells=CD45+B220-CD11c-TCRb+CD3+CD49b-CD4+(CD4+ T Cells) or CD8+(CD8+ T cells), yd T cells=CD45+B220-TCRb-CD49b-TCRyd+. FIG. 21B shows the proportion of adaptive immune cells that are CD103+XCR1+. FIG. 21C shows the proportion of CD103+XCR1+ adaptive immune cells that are CD62L−. FIG. 21D shows the proportion of adaptive immune cells that are CD103loXCR1−. FIG. 21E shows the proportion of CD103loXCR1− adaptive immune cells that are CD62L−. Control=black, ECM=teal, PE=pink. Data are means±95% Confidence Interval, n=3-5.

FIG. 22 shows expansion of active CD103+XCR1+ adaptive immune cell populations in the draining lymph node after injury. Cells isolated from inguinal lymph nodes. Top panel=proportion of cells that are CD103+XCR1+, Bottom panel=proportion of CD103+XCR1+ adaptive immune cells that are CD62L− (active). Uninjured=grey ⊗, Control injury=black ●. N=3 (uninjured), N=5 (control injury). All comparisons (Uninjured vs control injury) are significantly different, P<0.05, ANOVA with Tukey Post-hoc correction for multiple comparisons.

FIG. 23 shows CD44 and CD62L Expression on T cells. Representative FACS plot showing CD62L and CD44 expression on CD4+ T cells that are double positive for CD103 and XCR1 (blue) or double negative (grey).

FIGS. 24A-24C show CD103 and XCR1 expression on FoxP3+(FIG. 24A) and HELIOS+(FIGS. 24B & 24C) regulatory CD4 and CD8 T cells over time. Black=control injury, teal=ECM treated, pink=PE treated. Data are means±SEM, n=5.

FIG. 25 shows a comparison of CD103+XCR1+ adaptive immune cells in draining lymph node and peripheral blood at 7- and 21-days post injury. Proportion of adaptive immune cells that are positive for both XCR1 and CD103 in the draining (inguinal) lymph node (ILN) and peripheral blood. Data are means±SEM, n=4-5

FIGS. 26A-26D show up-regulation of Xcl1 and E-Cadherin in muscle is associated with tissue damage and material implantation. FIG. 26A shows Xcl1 mRNA relative quantification as fold change (2^{-ΔΔCt}) over uninjured control. FIG. 26B shows CD103 cells found at the material and injury interface, scale bar=75 um. FIG. 26C shows quantification of E-Cadherin expression via DAB quantification at the distal/uninjured muscle tissue, the injury interface, the material itself, and the external overlaying capsule. N/A=not applicable for control injury or uninjured sample. Data are means±SEM, n=3-5. FIG. 26D shows representative images of hematoxylin and eosin (H&E) and E-Cadherin (DAB) staining at the injury interface. Scale bars=300 um.

FIG. 27 shows cytokine and chemokine profile of local muscle tissue at 7 days post injury. Displayed as a fold change over uninjured control, Control (injury)=Black, ECM=teal, PE=pink. Representative of n=5 blots.

FIG. 28 shows a proposed mechanisms of tissue homeostasis and damage response through communication between CD103+XCR1+ innate and adaptive immune cells.

FIGS. 29A-29C shows that tolerogenic natural killer cells are recruited to tissue injury and induced by DAMPs. FIG. 29A: gene expression in cells isolated from muscle injury shows NK cells have high expression of Tgfb1 gene. TGFB-secreting NK cells are associated with a tolerogenic phenotype. FIG. 29B: shows that NK cells have >350-fold higher Xcl1 gene expression (the chemokine that binds XCR1) in comparison to other adaptive immune cells in the microenvironment. FIG. 29C shows Xcl1 expression levels in NK cells exposed in vitro to fragments of decellularized extracellular matrix (ECM) or low molecular weight hyaluronic acid (LMW-HA).

FIG. 30 illustrates a potential mechanism for recruitment of tolerogenic dendritic cells (DCs) to an injury space.

### DETAILED DESCRIPTION OF THE DISCLOSURE

The present disclosure relates to methods and compositions for improving the healing of wounds. More specifically, the present disclosure relates to compositions, and methods of using such compositions, that direct the immune response within a wound towards a pro-regenerative response. The disclosed compositions and methods are particularly useful in altering the immune response elicited in response to an implanted device, which often causes scarring and fibrosis at the site of implantation. Thus, a method of the disclosure may generally be practiced by introducing at the site of a wound a composition of the disclosure that induces a pro-regenerative environment within the wound. In certain aspects, the pro-regenerative environment comprises novel dendritic cells described herein.

Before the present disclosure is further described, it is to be understood that the disclosure is not limited to particular embodiments described, as such may, of course, vary. It is also to be understood that the terminology used herein is for the purpose of describing particular embodiments only, and is not intended to be limiting, since the scope of the present disclosure will be limited only by the claims.

It must be noted that as used herein and in the appended claims, the singular forms “a,” “an,” and “the” include plural referents unless the context clearly dictates otherwise. For example, a compound refers to one or more compound molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Publications disclosed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed. All publications mentioned herein are incorporated herein by reference to disclose and describe the methods and/or materials in connection with which the publications are cited.

Unless defined otherwise, all technical and scientific terms used herein have the same meaning as commonly understood by one of ordinary skill in the art to which the disclosure belongs. Although any methods and materials similar or equivalent to those described herein can also be used in the practice or testing of the present disclosure, the preferred methods and materials are now described. Terms and phrases, which are common to the various aspects disclosed herein, are defined below.

Wound healing comprises a variety of growth factors and cytokines that regulate cell growth, cell differentiation, and cell proliferation. While wound healing generally occurs in phases, these phases may overlap to some extent. Following tissue injury, epithelial and/or endothelial cells release inflammatory mediators that initiate an antifibrinolytic-coagulation cascade, which triggers blood clot formation. This is followed by an inflammatory and proliferative phase in which leukocytes are recruited and then activated and induced to proliferate by chemokines and growth factors. The activated leukocytes secrete profibrotic cytokines such as IL-13 and TGF-b. Stimulated epithelial cells, endothelial cells, and myofibroblasts produce matrix metalloproteinases (MMPs), which disrupt the basement membrane, and additional cytokines and chemokines that recruit and activate neutrophils, macrophages, T cells, B cells, and eosinophils, important components of tissue regeneration. The activated macrophages and neutrophils clean up tissue debris, dead cells, and invading organisms. Shortly after the initial inflammatory phase, myofibroblasts produce ECM components, and endothelial cells form new blood vessels. The myofibroblasts may be derived from local mesenchymal cells, recruited from the bone marrow (where they are known as fibrocytes), or they may be derived by epithelial-mesenchymal transition (EMT). In the subsequent remodeling and maturation phase, the activated myofibroblasts stimulate wound contraction. Collagen fibers also become more organized, blood vessels are restored to normal, scar tissue is eliminated, and epithelial and/or endothelial cells divide and migrate over the basal layers to regenerate the epithelium or endothelium, respectively, restoring the damaged tissue to its normal appearance. The aforementioned process generally described a pro-regenerative response to a wound. However, in certain instances, for example chronic wounds, the normal healing process is disrupted. Persistent inflammation, tissue necrosis, and infection lead to chronic myofibroblast activation and excessive accumulation of ECM components, which promotes the formation of a permanent fibrotic scar. However, the inventors have discovered that by treating the wound with appropriate compositions, the immune response within a wound may be directed away from a fibrotic response, and towards a pro-regenerative response.

One aspect of the disclosure is a method, comprising administering at the site of a wound a therapeutic composition that induces a pro-regenerative environment within the wound and/or within tissue surrounding the wound.

The term “wound” refers to damage to the integrity of biological tissue, such as skin (including the epidermis, dermis, and hypodermis), mucous membranes, and organ tissues (e.g., muscle, lung tissue, etc.). Wounds suitable for use of methods of the disclosure may be closed wounds, or they may be open wounds. Wounds of the present disclosure include, but are not limited to, burns, contusions, seromas, hematomas, lacerations, avulsions, punctures, surgical wounds (e.g., an incision), ulcers and wounds due to crushing injuries. Thus, in one aspect, the wound is selected from the group consisting of a burn, a contusion, a seroma, a hematoma, a laceration, an avulsion, a puncture, a surgical wound (e.g., an incision), an ulcer and a wound due to a crushing injury.

“Administering at the site of a wound”, “applying to the wound”, and the like, mean introducing the therapeutic composition into the cavity of the wound (e.g., an incision), and/or into the tissue surrounding the wound cavity. It is well understood that while the most visible sign of a wound such as an incision is the resulting open cavity, the tissue surround the cavity is also affected, and infiltration and proliferation of immune cells with in such tissue is instrumental in healing of the wound. Thus, in certain aspects, introduction of the therapeutic composition may comprise, or may be exclusive to, tissue surrounding the wound cavity. In certain aspects, the location of introduction may be determined based on the physical characteristics of the therapeutic composition. For example, if the composition is a cream or a foam, it may be best if the composition is applied to the surface of the skin in the cavity of the wound. Alternatively, if the composition is a liquid, it may be applied to the skin in the wound cavity and/or it may be injected in the tissue within or surrounding the wound cavity.

A “therapeutic composition” is a composition comprising a therapeutic agent that induces a pro-regenerative environment at, and surrounding, a location at which the therapeutic composition is administered to an individual. A therapeutic composition of the disclosure may comprise any formulation suitable for delivery of the therapeutic agent so that the therapeutic agent is able to induce a pro-regenerative environment. In certain aspects, the therapeutic composition may be formulated as an ointment, a spray, a lotion, a gel, a cream, a foam, a solution, a suspension, an emulsion, a hydrogel, or a paste. In certain aspects, the therapeutic composition may comprise liposomes, microspheres or nanoparticles.

In certain aspects, a therapeutic composition may comprise t least one additional agent (e.g., a pharmacologically acceptable excipient), such as a buffer, a stabilizing agent, a chelator, an antioxidant, a preservative, and any mixture(s) thereof.

In certain aspects, the therapeutic composition may be formulated as a slow-release composition. A slow-release composition is a composition that releases an active ingredient (e.g., therapeutic agent) slowly over a period of time instead of all at once. Methods of making slow-release compositions are known to those of skill in the art.

A “therapeutic agent” refers to a molecule or combination of molecules that, when administered to an individual, induces a pro-regenerative response at, and preferably proximal to, a location at which the therapeutic composition is administered to an individual. A therapeutic agent may be any type of molecule that is able to induce a pro-regenerative environment. In certain aspects, the therapeutic agent may comprise a peptide, a protein, a glycoprotein, a lipoprotein, a lipid, a sugar, a polysaccharide, a glycosaminoglycan, a nucleic acid molecule, an organic molecule, or any combination(s) thereof. Thus, a therapeutic agent may comprise one or more than one type of molecule. A therapeutic agent of the disclosure may be isolated from biological material (e.g., cells, organism, etc.), it may be produced using recombinant DNA technology, it may be synthesized chemically, or it may be produced using a combination of such technologies. The term “isolated” does not denote a particular degree of isolation.

In certain aspects, the therapeutic agent may comprise decellularized extracellular matrix (ECM), or one or more components derived therefrom. ECM is the non-cellular portion of tissues and organs and comprises a network of proteins (e.g., collagen, elastin, laminin) and other molecules such as proteoglycans and polysaccharides. Following injury, enzymes (particularly MMPs) are activated, and these activated enzymes degrade proteins of the ECM, such as collagen and elastin. These degradation products act to, among other things, restrict migration of stromal cells and accelerate fibroblast proliferation. Thus, in certain aspects, the therapeutic agent may comprise one or more components of ECM, or one or more components derived therefrom. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, and agrin.

Dynamic remodeling of the ECM is required for wound healing. During remodeling, the ECM undergoes proteolytic processing that releases bioactive matrix fragments, which have been termed “matrikines”. Examples of matrikines are disclosed in WO2022/055974, which is incorporated herein by reference in its entirety. Matrikines have been shown to promote cellular infiltration, progressive tissue damage, or wound healing. Thus, these signals represent an important effector mechanism of the ECM for cell signaling and trafficking of cells into target organs. In certain aspects, the therapeutic agent may comprise one or more degradation products of ECM. In certain aspects, the therapeutic agent comprises a matrikine. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, and agrin. In certain aspects, the therapeutic agent comprise a matrikine selected from the group consisting of metastatin, affesten, canstatin, tetrastatin, pentastatin, lamstatin, hexastatin, endotrophin, restin1, restin2, restin3, restin4, endostatin, neostatin, anastellin, sibsttin, PEX, endorepellin, CUB1CUB2 domain, Ten/2, Ten11/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin, and any combination thereof.

In certain aspects, the therapeutic agent may comprise one or more damage associated molecular patterns (DAMPs). Damage-associated molecular patterns (DAMPs) are endogenous danger molecules that are released from damaged or dying cells and activate the innate immune system by, for example, interacting with pattern recognition receptors (PRRs). DAMPs can originate from different sources and include, for example, extracellular proteins, such as biglycan and tenascin C, intracellular proteins, such as high-mobility group box 1 (HMGB1), histones, S100 proteins, heat-shock proteins (HSPs), and plasma proteins, like fibrinogen, Gc-globulin, and serum amyloid A (SAA). Examples of DAMPs included, but are not limited to, biglycan, decorin, versican, LMW hyaluronan, heparin sulfate, fibronectin, including the EDA domain, fibrinogen, tenascin C, uric acid, 5100 proteins, heat shock proteins, adenosine triphosphate (ATP), F-actin, cyclophilin A, amyloid beta (A3), histones, HMGB1, HMGN1, IL-la, IL-33, SAP130, deoxyribonucleic acid (DNA), ribonucleic acid (RNA), mitochondrial DNA (mtDNA), transcription factor A mitochondrial (TFAM), formyl peptide, mitochondrial reactive oxygen species (mROS), calreticulin, defensins, calthelicidin (LL37), eosinophil-derived neurotoxins, granulysin, syndecans, and glypicans.

In some aspects, the therapeutic agent may comprise a compound that induces the production or release of an ECM component, or one or more component therefrom, a matrikine, or a DAMP.

As used herein, a “pro-regenerative environment” refers to a composition of factors (e.g., immune cells, cytokines, etc.) within and surrounding a wound that direct the healing process away from fibrosis and that promote regeneration of tissue within the wound, so that the damaged tissue is restored to its normal appearance. As discussed above, injury initiates a cascade of events that triggers remodeling of ECM and tissue and the mobilization of cells into the wound site to initiate host defense and tissue repair. Following injury, some of the earliest cells to respond are polymorphonuclear cells, including neutrophils, eosinophils, and basophils. Neutrophils are phagocytic cells that scavenge debris and produce the recruitment of other cells, such as macrophages. Thus, a varied set of immune cells is recruited to the site of injury in the first few days following injury. In certain aspects, the therapeutic agent induces a pro-generative environment, which may comprise a Th2-driven immune environment. A TH2 environment is characterized by the presence of eosinophils, basophils, mast cell degranulation, and M2 macrophages, the latter of which is associated with wound healing and repair. In addition, a Th2 environment exhibits increased levels of one or more cytokines, such as inerleukin-4 (IL-4), IL-5, IL-10, and IL-13, which are important for the induction of humoral immune responses. In certain aspects, the Th2-driven environment may comprise an eosinophil-dominant granulocytic compartment at the site of injury. In certain aspects, a therapeutic agent of the disclosure may induce an influx of macrophages having an M2 phenotype. In certain aspects, a therapeutic agent of the disclosure may induce local proliferation of M2 macrophages at the site of injury. In certain aspects, a therapeutic agent of the disclosure may induce differentiation of a macrophage into a M2 macrophages. In certain aspects, M2 macrophages within the Th2-driven environment may comprise high levels of CD206, CD301b, and CD169. Such levels are indicative of local proliferation of tissue-resident cells. Thus, therapeutic agents of the disclosure may induce an increase in the number of M2 macrophages having high levels of CD206, CD301b, and CD169 within the wound.

The second most common antigen presenting cell (APC) found within the wound environment following injury is a dendritic cell, such as a CD11c⁺CD11b^{lo/neg} dendritic cell. The inventors have discovered that therapeutic compositions of the disclosure induce pro-regenerative environments containing an increased number of conventional dendritic cells (cDC1s). cDC1 cells can initiate de novo T cell responses, as well as attract T cells, secrete cytokines, and enhance local cytotoxic T cell function. cDC1 cells can also induce tolerance. In certain aspects, therapeutic agents of the disclosure may induce an increase in the number of conventional dendritic cells (cDC1s). Such cells may be cross-presenting dendritic cells, which may be XCR1⁺CD103⁺dendritic cells. In certain aspects, therapeutic compositions of the disclosure induce an influx of XCR1+CD103+cDC1s. In certain aspects, therapeutic compositions of the disclosure induce local proliferation of XCR1+CD103+ cDC1s. In certain aspects, therapeutic compositions of the disclosure induce differentiation of a dendritic cell into a XCR1+CD103+ cDC1.

In certain aspects, the therapeutic agent may induce a therapeutic environment that may comprise an increase in NK cells. Such increase may be due to increased recruitment of NK cells to the site of the therapeutic agent, or it may be due to increased replication of NK cells. In certain aspects, such NK cells may be CD49b+TCRβ−. In certain aspects, such NK cells may exhibit up-regulation of Xcl1 gene expression, which may be induced by the therapeutic agent.

One aspect of the disclosure is a method of altering an immune response to a wound in an individual, comprising administering at the site of the wound a therapeutic composition comprising a therapeutic agent that induces a pro-regenerative environment within the wound and/or within tissue surrounding the wound, thereby altering the immune response to the wound. In certain aspects, the wound may comprise a burn, a contusion, a seroma, a hematoma, a laceration, an avulsion, a puncture, a surgical wound, an incision, an ulcer and a wound due to a crushing injury. In certain aspects, the therapeutic composition may comprise an ointment, a spray, a lotion, a gel, a cream, a foam, a solution, a suspension, an emulsion, a hydrogel, or a paste. In certain aspects, the therapeutic composition may comprise liposomes, microspheres or nanoparticles. In certain aspects, the therapeutic agent may induce a pro-regenerative immune response within the wound or within tissue proximal to the wound. In certain aspects, the pro-regenerative immune response may comprise an eosinophil-dominant granulocytic compartment. In certain aspects, the therapeutic agent may induce a TH2-driven immune response. In certain aspects, the therapeutic agent may induce an influx of M2 macrophages into the wound. In certain aspects, the therapeutic agent may induce a local proliferation of M2 macrophages within the wound. In certain aspects, the therapeutic agent may induce a macrophage to differentiate into an M2 macrophage. In certain aspects, the Th2-driven immune response may comprise macrophages having high levels of CD206, CD301b, and/or CD169. In certain aspects, the therapeutic agent may induce local proliferation of tissue resident macrophages having high levels of CD206, CD301b, and/or CD169. In certain aspects, the therapeutic agent may cause enrichment of cross-presenting dendritic cells in the wound or in tissue proximal to the wound. In certain aspects, the therapeutic agent may induce an influx of the cross-presenting dendritic cells. In certain aspects, the therapeutic agent may induce local proliferation of the cross-presenting dendritic cells. In certain aspects, the therapeutic agent may induce a dendritic cell to differentiate into a cross-presenting dendritic cell. In certain aspects, the cross-presenting dendritic cells may comprise cDC1 cells. In certain aspects, the cross-presenting dendritic cells may be XCR1+CD103+ dendritic cells. In certain aspects, the cross-presenting dendritic cells may express intermediate levs of CD86. In certain aspects, the therapeutic agent may induce an increase in CD44⁺CD26L⁻ T cells in the individual. In certain aspects, the CD44⁺CD26L⁻ T cells may comprise CD4+ cells. In certain aspects, the CD44⁺CD26L⁻ T cells may comprise CD8+ cells. In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, and agrin. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin, and any combination thereof. In certain aspects, the therapeutic agent comprise a matrikine selected from the group consisting of metastatin, affesten, canstatin, tetrastatin, pentastatin, lamstatin, hexastatin, endotrophin, restini, restin2, restin3, restin4, endostatin, neostatin, anastellin, sibsttin, PEX, endorepellin, CUB1CUB2 domain, Ten/2, Teni1/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin and any combination thereof. In certain aspects, the therapeutic agent may comprise one or more DAMPs.

One aspect of the disclosure is a method of treating an individual having a wound, comprising administering at the site of the wound a therapeutic composition comprising a therapeutic agent that induces a pro-regenerative environment within the wound and/or within tissue surrounding the wound, thereby treating the individual. In certain aspects, the wound may comprise a burn, a contusion, a seroma, a hematoma, a laceration, an avulsion, a puncture, a surgical wound, an incision, an ulcer and a wound due to a crushing injury. In certain aspects, the therapeutic composition may comprise an ointment, a spray, a lotion, a gel, a cream, a foam, a solution, a suspension, an emulsion, a hydrogel, or a paste. In certain aspects, the therapeutic composition may comprise liposomes, microspheres or nanoparticles. In certain aspects, the therapeutic agent may induce a pro-regenerative immune response within the wound or within tissue proximal to the wound. In certain aspects, the pro-regenerative immune response may comprise an eosinophil-dominant granulocytic compartment. In certain aspects, the therapeutic agent may induce a TH2-driven immune response. In certain aspects, the therapeutic agent may induce an influx of M2 macrophages into the wound. In certain aspects, the therapeutic agent may induce an local proliferation of M2 macrophages within the wound. In certain aspects, the therapeutic agent may induce a macrophage to differentiate into an M2 macrophage. In certain aspects, the Th2-driven immune response may comprise macrophages having high levels of CD206, CD301b, and/or CD169. In certain aspects, the therapeutic agent may induce local proliferation of tissue resident macrophages having high levels of CD206, CD301b, and/or CD169. In certain aspects, the therapeutic agent may cause enrichment of NK cells in the wound or in tissue proximal to the wound. Such enrichment may result from increased recruitment of NK cells or from increased local proliferation of NK cells. In certain aspects, such NK cells may be CD49b+ TCRβ−. In certain aspects, such NK cells may exhibit up-regulation of Xcl1 gene expression, which may be induced by the therapeutic agent.

In certain aspects, the therapeutic agent may cause enrichment of cross-presenting dendritic cells in the wound or in tissue proximal to the wound. In certain aspects, the therapeutic agent may induce an influx of the cross-presenting dendritic cells. In certain aspects, the therapeutic agent may induce local proliferation of the cross-presenting dendritic cells. In certain aspects, the therapeutic agent may induce a dendritic cell to differentiate into a cross-presenting dendritic cell. In certain aspects, the cross-presenting dendritic cells may comprise cDC1 cells. In certain aspects, the cross-presenting dendritic cells may be XCR1+CD103+ dendritic cells. In certain aspects, the cross-presenting dendritic cells may express intermediate levs of CD86. In certain aspects, the therapeutic agent may induce an increase in CD44⁺CD26L⁻ T cells in the individual. In certain aspects, the CD44⁺CD26L⁻ T cells may comprise CD4+ cells. In certain aspects, the CD44⁺CD26L⁻ T cells may comprise CD8+ cells. In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin, and any combination thereof. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin, and any combination thereof. In certain aspects, the therapeutic agent comprise a matrikine selected from the group consisting of metastatin, affesten, canstatin, tetrastatin, pentastatin, lamstatin, hexastatin, endotrophin, restini, restin2, restin3, restin4, endostatin, neostatin, anastellin, sibsttin, PEX, endorepellin, CUB1CUB2 domain, Ten/2, Teni1/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin and any combination thereof. In certain aspects, the therapeutic agent may comprise one or more DAMPs.

One aspect of the disclosure is a method of treating a wound in an individual, comprising administering at the site of the wound a therapeutic composition comprising a therapeutic agent that induces a pro-regenerative environment within the wound and/or within tissue surrounding the wound, thereby treating the wound. In certain aspects, the wound may comprise a burn, a contusion, a seroma, a hematoma, a laceration, an avulsion, a puncture, a surgical wound, an incision, an ulcer and a wound due to a crushing injury. In certain aspects, the therapeutic composition may comprise an ointment, a spray, a lotion, a gel, a cream, a foam, a solution, a suspension, an emulsion, a hydrogel, or a paste. In certain aspects, the therapeutic composition may comprise liposomes, microspheres or nanoparticles. In certain aspects, the therapeutic agent may induce a pro-regenerative immune response within the wound or within tissue proximal to the wound. In certain aspects, the pro-regenerative immune response may comprise an eosinophil-dominant granulocytic compartment. In certain aspects, the therapeutic agent may induce a TH2-driven immune response. In certain aspects, the therapeutic agent may induce an influx of M2 macrophages into the wound. In certain aspects, the therapeutic agent may induce an local proliferation of M2 macrophages within the wound. In certain aspects, the therapeutic agent may induce a macrophage to differentiate into an M2 macrophage. In certain aspects, the Th2-driven immune response may comprise macrophages having high levels of CD206, CD301b, and/or CD169. In certain aspects, the therapeutic agent may induce local proliferation of tissue resident macrophages having high levels of CD206, CD301b, and/or CD169. In certain aspects, the therapeutic agent may cause enrichment of NK cells in the wound or in tissue proximal to the wound. Such enrichment may result from increased recruitment of NK cells or from increased local proliferation of NK cells. In certain aspects, such NK cells may be CD49b+ TCRβ−. In certain aspects, such NK cells may exhibit up-regulation of Xcl1 gene expression, which may be induced by the therapeutic agent. In certain aspects, the therapeutic agent may cause enrichment of cross-presenting dendritic cells in the wound or in tissue proximal to the wound. In certain aspects, the therapeutic agent may induce an influx of the cross-presenting dendritic cells. In certain aspects, the therapeutic agent may induce local proliferation of the cross-presenting dendritic cells. In certain aspects, the therapeutic agent may induce a dendritic cell to differentiate into a cross-presenting dendritic cell. In certain aspects, the cross-presenting dendritic cells may comprise cDC1 cells. In certain aspects, the cross-presenting dendritic cells may be XCR1⁺CD103⁺dendritic cells. In certain aspects, the cross-presenting dendritic cells may express intermediate levs of CD86. In certain aspects, the therapeutic agent may induce an increase in CD44⁺CD26L⁻ T cells in the individual. In certain aspects, the CD44⁺CD26L⁻ T cells may comprise CD4+ cells. In certain aspects, the CD44⁺CD26L⁻ T cells may comprise CD8+ cells. In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, and agrin. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, and agrin. In certain aspects, the therapeutic agent comprise a matrikine selected from the group consisting of metastatin, affesten, canstatin, tetrastatin, pentastatin, lamstatin, hexastatin, endotrophin, restin1, restin2, restin3, restin4, endostatin, neostatin, anastellin, sibsttin, PEX, endorepellin, CUB1CUB2 domain, Ten/2, Ten11/12/13, Ten14, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, and tumstatin. In certain aspects, the therapeutic agent may comprise one or more DAMPs.

One aspect of the disclosure is a method of implanting a medical device in an individual, comprising introducing the medical device into tissue within the individual, and administering at the site of the implanted medical device a therapeutic composition comprising a therapeutic agent that induces a pro-regenerative environment within the wound and/or within tissue surrounding the wound. In certain aspects, the wound may comprise a burn, a contusion, a seroma, a hematoma, a laceration, an avulsion, a puncture, a surgical wound, an incision, an ulcer and a wound due to a crushing injury. In certain aspects, the therapeutic composition may comprise an ointment, a spray, a lotion, a gel, a cream, a foam, a solution, a suspension, an emulsion, a hydrogel, or a paste. In certain aspects, the therapeutic composition may comprise liposomes, microspheres or nanoparticles. In certain aspects, the therapeutic agent may induce a pro-regenerative immune response within the wound or within tissue proximal to the wound. In certain aspects, the pro-regenerative immune response may comprise an eosinophil-dominant granulocytic compartment. In certain aspects, the therapeutic agent may induce a TH2-driven immune response. In certain aspects, the therapeutic agent may induce an influx of M2 macrophages into the wound. In certain aspects, the therapeutic agent may induce an local proliferation of M2 macrophages within the wound. In certain aspects, the therapeutic agent may induce a macrophage to differentiate into an M2 macrophage. In certain aspects, the Th2-driven immune response may comprise macrophages having high levels of CD206, CD301b, and/or CD169. In certain aspects, the therapeutic agent may induce local proliferation of tissue resident macrophages having high levels of CD206, CD301b, and/or CD169. In certain aspects, the therapeutic agent may cause enrichment of NK cells in the wound or in tissue proximal to the wound. Such enrichment may result from increased recruitment of NK cells or from increased local proliferation of NK cells. In certain aspects, such NK cells may be CD49b+ TCRβ−. In certain aspects, such NK cells may exhibit up-regulation of Xcl1 gene expression, which may be induced by the therapeutic agent. In certain aspects, the therapeutic agent may cause enrichment of cross-presenting dendritic cells in the wound or in tissue proximal to the wound. In certain aspects, the therapeutic agent may induce an influx of the cross-presenting dendritic cells. In certain aspects, the therapeutic agent may induce local proliferation of the cross-presenting dendritic cells. In certain aspects, the therapeutic agent may induce a dendritic cell to differentiate into a cross-presenting dendritic cell. In certain aspects, the cross-presenting dendritic cells may comprise cDC1 cells. In certain aspects, the cross-presenting dendritic cells may be XCR1+CD103+ dendritic cells. In certain aspects, the cross-presenting dendritic cells may express intermediate levs of CD86. In certain aspects, the therapeutic agent may induce an increase in CD44⁺CD26L⁻ T cells in the individual. In certain aspects, the CD44⁺CD26L⁻ T cells may comprise CD4+ cells. In certain aspects, the CD44⁺CD26L⁻ T cells may comprise CD8+ cells. In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin, and any combination thereof. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin, and any combination thereof. In certain aspects, the therapeutic agent comprise a matrikine selected from the group consisting of metastatin, affesten, canstatin, tetrastatin, pentastatin, lamstatin, hexastatin, endotrophin, restini, restin2, restin3, restin4, endostatin, neostatin, anastellin, sibsttin, PEX, endorepellin, CUB1CUB2 domain, Ten/2, Teni1/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin, and any combination thereof. In certain aspects, the therapeutic agent may comprise one or more DAMPs. A “medical device” refers to any device implanted in an individual for the purpose of improving the health and/or functioning of the individual. One example of a medical device is an implant. Examples of implants include, but are not limited to, breast implants, stents, ports, shunts, hip implants, knee implants, cochlear implants, hernia surgical mesh implants, intraocular lens implants, pacemakers, metal/surgical screws, metal/surgical rods, metal/surgical pins, artificial discs, and spinal fusion hardware. Such implants may be made using, for example, metal and metal alloys, plastic polymers, ceramics, hydrogels and composites, which may include, but which are not limited to, silicone, polyethylene, stainless steel, titanium, zirconia, polyurethane foam, polylactic acid, amalgam, gold, alumina, silicate, chrome, cobalt, and molybdenum.

One aspect of the disclosure is a method of reducing, or preventing, fibrosis in a wound, comprising administering at the site of the wound a therapeutic composition comprising a therapeutic agent that induces a pro-regenerative environment within the wound and/or within tissue surrounding the wound, thereby reducing, or preventing, fibrosis in the wound. In certain aspects, the wound may comprise a burn, a contusion, a seroma, a hematoma, a laceration, an avulsion, a puncture, a surgical wound, an incision, an ulcer and a wound due to a crushing injury. In certain aspects, the therapeutic composition may comprise an ointment, a spray, a lotion, a gel, a cream, a foam, a solution, a suspension, an emulsion, a hydrogel, or a paste. In certain aspects, the therapeutic composition may comprise liposomes, microspheres or nanoparticles. In certain aspects, the therapeutic agent may induce a pro-regenerative immune response within the wound or within tissue proximal to the wound. In certain aspects, the pro-regenerative immune response may comprise an eosinophil-dominant granulocytic compartment. In certain aspects, the therapeutic agent may induce a TH2-driven immune response. In certain aspects, the therapeutic agent may induce an influx of M2 macrophages into the wound. In certain aspects, the therapeutic agent may induce an local proliferation of M2 macrophages within the wound. In certain aspects, the therapeutic agent may induce a macrophage to differentiate into an M2 macrophage. In certain aspects, the Th2-driven immune response may comprise macrophages having high levels of CD206, CD301b, and/or CD169. In certain aspects, the therapeutic agent may induce local proliferation of tissue resident macrophages having high levels of CD206, CD301b, and/or CD169. In certain aspects, the therapeutic agent may cause enrichment of NK cells in the wound or in tissue proximal to the wound. Such enrichment may result from increased recruitment of NK cells or from increased local proliferation of NK cells. In certain aspects, such NK cells may be CD49b+ TCRβ−. In certain aspects, such NK cells may exhibit up-regulation of Xcl1 gene expression, which may be induced by the therapeutic agent. In certain aspects, the therapeutic agent may cause enrichment of cross-presenting dendritic cells in the wound or in tissue proximal to the wound. In certain aspects, the therapeutic agent may induce an influx of the cross-presenting dendritic cells. In certain aspects, the therapeutic agent may induce local proliferation of the cross-presenting dendritic cells. In certain aspects, the therapeutic agent may induce a dendritic cell to differentiate into a cross-presenting dendritic cell. In certain aspects, the cross-presenting dendritic cells may comprise cDC1 cells. In certain aspects, the cross-presenting dendritic cells may be XCR1+CD103+ dendritic cells. In certain aspects, the cross-presenting dendritic cells may express intermediate levs of CD86. In certain aspects, the therapeutic agent may induce an increase in CD44⁺CD26L⁻ T cells in the individual. In certain aspects, the CD44⁺CD26L⁻ T cells may comprise CD4+ cells. In certain aspects, the CD44⁺CD26L⁻ T cells may comprise CD8+ cells. In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin, and any combination thereof. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin, and any combination thereof. In certain aspects, the therapeutic agent comprise a matrikine selected from the group consisting of metastatin, affesten, canstatin, tetrastatin, pentastatin, lamstatin, hexastatin, endotrophin, restini, restin2, restin3, restin4, endostatin, neostatin, anastellin, sibsttin, PEX, endorepellin, CUB1CUB2 domain, Ten/2, Teni1/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin, and any combination thereof. In certain aspects, the therapeutic agent may comprise one or more DAMPs.

One aspect of the disclosure is a therapeutic composition for treating a wound, the composition comprising a therapeutic agent that induces a pro-regenerative environment within the wound and/or within tissue surrounding the wound. In certain aspects, the wound may comprise a burn, a contusion, a seroma, a hematoma, a laceration, an avulsion, a puncture, a surgical wound, an incision, an ulcer and a wound due to a crushing injury. In certain aspects, the therapeutic composition may comprise an ointment, a spray, a lotion, a gel, a cream, a foam, a solution, a suspension, an emulsion, a hydrogel, or a paste. In certain aspects, the therapeutic composition may comprise liposomes, microspheres or nanoparticles. In certain aspects, the therapeutic agent may induce a pro-regenerative immune response within the wound or within tissue proximal to the wound. In certain aspects, the pro-regenerative immune response may comprise an eosinophil-dominant granulocytic compartment. In certain aspects, the therapeutic agent may induce a TH2-driven immune response. In certain aspects, the therapeutic agent may induce an influx of M2 macrophages into the wound. In certain aspects, the therapeutic agent may induce an local proliferation of M2 macrophages within the wound. In certain aspects, the therapeutic agent may induce a macrophage to differentiate into an M2 macrophage. In certain aspects, the Th2-driven immune response may comprise macrophages having high levels of CD206, CD301b, and/or CD169. In certain aspects, the therapeutic agent may induce local proliferation of tissue resident macrophages having high levels of CD206, CD301b, and/or CD169. In certain aspects, the therapeutic agent may cause enrichment of NK cells in the wound or in tissue proximal to the wound. Such enrichment may result from increased recruitment of NK cells or from increased local proliferation of NK cells. In certain aspects, such NK cells may be CD49b+ TCRβ−. In certain aspects, such NK cells may exhibit up-regulation of Xcl1 gene expression, which may be induced by the therapeutic agent. In certain aspects, the therapeutic agent may cause enrichment of cross-presenting dendritic cells in the wound or in tissue proximal to the wound. In certain aspects, the therapeutic agent may induce an influx of the cross-presenting dendritic cells. In certain aspects, the therapeutic agent may induce local proliferation of the cross-presenting dendritic cells. In certain aspects, the therapeutic agent may induce a dendritic cell to differentiate into a cross-presenting dendritic cell. In certain aspects, the cross-presenting dendritic cells may comprise cDC1 cells. In certain aspects, the cross-presenting dendritic cells may be XCR1+CD103+ dendritic cells. In certain aspects, the cross-presenting dendritic cells may express intermediate levs of CD86. In certain aspects, the therapeutic agent may induce an increase in CD44⁺CD26L⁻ T cells in the individual. In certain aspects, the CD44⁺CD26L⁻ T cells may comprise CD4+ cells. In certain aspects, the CD44⁺CD26L⁻ T cells may comprise CD8+ cells. In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin, and any combination thereof. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin, and any combination thereof. In certain aspects, the therapeutic agent comprise a matrikine selected from the group consisting of metastatin, affesten, canstatin, tetrastatin, pentastatin, lamstatin, hexastatin, endotrophin, restin1, restin2, restin3, restin4, endostatin, neostatin, anastellin, sibsttin, PEX, endorepellin, CUB1CUB2 domain, Ten/2, Ten11/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin, and any combination thereof. In certain aspects, the therapeutic agent may comprise one or more DAMPs.

One aspect of the disclosure is a method of recruiting NK cells to a wound, or to tissue proximal to a wound, comprising administering at the site of the wound a therapeutic composition comprising a therapeutic agent that induces a pro-regenerative environment within the wound and/or within tissue surrounding the wound, thereby recruiting NK cells to the wound or to tissue proximal to the wound. The wound may comprise a burn, a contusion, a seroma, a hematoma, a laceration, an avulsion, a puncture, a surgical wound, an incision, an ulcer and a wound due to a crushing injury. In certain aspects, the therapeutic composition may comprise an ointment, a spray, a lotion, a gel, a cream, a foam, a solution, a suspension, an emulsion, a hydrogel, or a paste. In certain aspects, the therapeutic composition may comprise liposomes, microspheres or nanoparticles. In certain aspects, the therapeutic agent may induce increased recruitment of NK cells to the wound or the tissue proximal to the wound. In certain aspects, the therapeutic agent may induce increased local proliferation of NK cells. In certain aspects, the NK cells may be CD49b+ TCRβ−. In certain aspects, the NK cells may exhibit up-regulation of Xcl1 gene expression, which may be induced by the therapeutic agent. In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin, and any combination thereof. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin, and any combination thereof. In certain aspects, the therapeutic agent comprise a matrikine selected from the group consisting of metastatin, affesten, canstatin, tetrastatin, pentastatin, lamstatin, hexastatin, endotrophin, restin1, restin2, restin3, restin4, endostatin, neostatin, anastellin, sibsttin, PEX, endorepellin, CUB1CUB2 domain, Ten/2, Ten11/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin, and any combination thereof. In certain aspects, the therapeutic agent may comprise one or more DAMPs.

One aspect of the disclosure is a method of activating an NK cell, comprising contacting the NK cell with therapeutic agent of the disclosure. In certain aspects, the therapeutic agent causes replication of the NK cell. In certain aspects, the therapeutic agent cause migration of the NK cell. In certain aspects, the therapeutic agent increases Xcl1 gene expression in the NK cell. In certain aspects, the therapeutic agent induces increased secretion of Xcl1 protein. In certain aspects, contacting comprising introducing the therapeutic agent into a wound or into tissue proximal to a wound. In certain aspects, contacting comprises introducing the therapeutic agent to an NK cell in vitro (e.g., tissue culture). In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin, and any combination thereof. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin, and any combination thereof. In certain aspects, the therapeutic agent comprise a matrikine selected from the group consisting of metastatin, affesten, canstatin, tetrastatin, pentastatin, lamstatin, hexastatin, endotrophin, restini, restin2, restin3, restin4, endostatin, neostatin, anastellin, sibsttin, PEX, endorepellin, CUB1CUB2 domain, Ten/2, Teni1/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin, and any combination thereof. In certain aspects, the therapeutic agent may comprise one or more DAMPs.

One aspect of the disclosure is a method of inducing increased expression of Xcl1 in an NK cell, comprising contacting the NK cell with therapeutic agent of the disclosure. In certain aspects, the therapeutic agent increases Xcl1 gene expression in the NK cell. In certain aspects, the therapeutic agent induces increased secretion of Xcl1 protein. In certain aspects, contacting comprising introducing the therapeutic agent into a wound or into tissue proximal to a wound. In certain aspects, contacting comprises introducing the therapeutic agent to an NK cell in vitro (e.g., tissue culture). In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin and any combination thereof. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin, and any combination thereof. In certain aspects, the therapeutic agent comprise a matrikine selected from the group consisting of metastatin, affesten, canstatin, tetrastatin, pentastatin, lamstatin, hexastatin, endotrophin, restini, restin2, restin3, restin4, endostatin, neostatin, anastellin, sibsttin, PEX, endorepellin, CUB1CUB2 domain, Ten/2, Teni1/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin, and any combination thereof. In certain aspects, the therapeutic agent may comprise one or more DAMPs.

A kit comprising for treating a wound, the kit comprising, at least, a therapeutic composition comprising a therapeutic agent that induces a pro-regenerative environment within the wound and/or within tissue surrounding the wound. In certain aspects, the wound may comprise a burn, a contusion, a seroma, a hematoma, a laceration, an avulsion, a puncture, a surgical wound, an incision, an ulcer and a wound due to a crushing injury. In certain aspects, the therapeutic composition may comprise an ointment, a spray, a lotion, a gel, a cream, a foam, a solution, a suspension, an emulsion, a hydrogel, or a paste. In certain aspects, the therapeutic composition may comprise liposomes, microspheres or nanoparticles. In certain aspects, the therapeutic agent may induce a pro-regenerative immune response within the wound or within tissue proximal to the wound. In certain aspects, the pro-regenerative immune response may comprise an eosinophil-dominant granulocytic compartment. In certain aspects, the therapeutic agent may induce a TH2-driven immune response. In certain aspects, the therapeutic agent may induce an influx of M2 macrophages into the wound. In certain aspects, the therapeutic agent may induce a local proliferation of M2 macrophages within the wound. In certain aspects, the therapeutic agent may induce a macrophage to differentiate into an M2 macrophage. In certain aspects, the Th2-driven immune response may comprise macrophages having high levels of CD206, CD301b, and/or CD169. In certain aspects, the therapeutic agent may induce local proliferation of tissue resident macrophages having high levels of CD206, CD301b, and/or CD169. In certain aspects, the therapeutic agent may cause enrichment of NK cells in the wound or in tissue proximal to the wound. Such enrichment may result from increased recruitment of NK cells or from increased local proliferation of NK cells. In certain aspects, such NK cells may be CD49b+ TCRβ−. In certain aspects, such NK cells may exhibit up-regulation of Xcl1 gene expression, which may be induced by the therapeutic agent. In certain aspects, the therapeutic agent may cause enrichment of cross-presenting dendritic cells in the wound or in tissue proximal to the wound. In certain aspects, the therapeutic agent may induce an influx of the cross-presenting dendritic cells. In certain aspects, the therapeutic agent may induce local proliferation of the cross-presenting dendritic cells. In certain aspects, the therapeutic agent may induce a dendritic cell to differentiate into a cross-presenting dendritic cell. In certain aspects, the cross-presenting dendritic cells may comprise cDC1 cells. In certain aspects, the cross-presenting dendritic cells may be XCR1⁺CD103⁺dendritic cells. In certain aspects, the cross-presenting dendritic cells may express intermediate levs of CD86. In certain aspects, the therapeutic agent may induce an increase in CD44⁺CD26L⁻ T cells in the individual. In certain aspects, the CD44⁺CD26L⁻ T cells may comprise CD4+ cells. In certain aspects, the CD44⁺CD26L⁻ T cells may comprise CD8+ cells. In certain aspects, the therapeutic agent may comprise one or more components of ECM. In certain aspects, the therapeutic agent may comprise one or more components selected from the group consisting of collagen, laminin, fibronectin, elastin, chondroitin sulfate, heparan sulfate, keratan sulfate, hyaluronic acid, perlecan, agrin, and any combination thereof. In certain aspects, the therapeutic agent may comprise one or more degradation products from one or more components selected from the group consisting of a collagen, laminin, fibronectin, elastin, chondroitin sulfate, heparan sulfate, keratan sulfate, hyaluronic acid, perlecan, agrin and any combination thereof. In certain aspects, the therapeutic agent comprise a matrikine selected from the group consisting of metastatin, affesten, canstatin, tetrastatin, pentastatin, lamstatin, hexastatin, endotrophin, restini, restin2, restin3, restin4, endostatin, neostatin, anastellin, sibsttin, PEX, endorepellin, CUB1CUB2 domain, Ten/2, Ten11/12/13, Tenl4, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, tumstatin and any combination thereof. In certain aspects, the therapeutic agent may comprise one or more DAMPs. A kit of the disclosure may comprise additional components, such as needles, syringes, vials, applicators, and instructions for using components of the kit for treating a wound.

This written description uses examples to disclose the disclosure, including the best mode, and to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLES

### Materials and Methods

### Material Preparation

Small intestine was sourced from 5- to 6-month-old American Yorkshire pigs (Wagner Meats). The submucosa layer (SIS) was mechanically isolated by removal of the muscularis layer and subsequent mechanical scraping of the luminal layer. Resulting SIS was rinsed in distilled water and frozen at −800C until decellularization. After thawing, within a biosafety cabinet SIS was cut into 1-inch segments then incubated in 4% ethanol (Fisher Scientific) and 0.1% peracetic acid (Sigma) for 30 minutes with vigorous shaking or on a stir plate. Resulting decellularized ECM was neutralized with successive washes of sterile 1×PBS and distilled water. Liquid was blotted with sterile absorbent pads then material was transferred to a 50 ml conical tube and frozen at −80° C. until lyophilization for 48 hours. Dried material was loaded into sterile cryogenic milling containers and milled into a fine powder. The resulting powder was hydrated with sterile saline to form a thick paste that was then loaded into a slip-tip 1 ml syringe for application into the wound site.

Polyethylene powder (PE) particle size <150 m was purchased from Goodfellow Cambridge Limited and soaked in distilled water prior to rinsing with 70% ethanol and UV sterilized in ethanol for 30 minutes. PE was stored in 70% ethanol until use. PE particles suspended in ethanol were transferred to an Eppendorf tube and dried overnight in a biosafety cabinet. Due to the hydrophobicity of PE, samples cannot be loaded into a syringe and are applied directly to the wound as a powder.

### Volumetric Muscle Loss Surgery

Mice received bilateral volumetric muscle loss trauma as per previously described method (1). Briefly, the lower limbs of 6-8-week-old female C57BL/6 WT mice (Jackson laboratory) were shaved with electric razor and cleared of excess hair by depilatory cream, one day before surgery. Next day, mice were anesthetized in anesthesia chamber under 4.0% isoflurane in oxygen at a 200 cc/min flow rate and received subcutaneous injection of buprenorphine for pain management. The mice were then maintained at 2.0% isoflurane for the duration of the procedure. The site of surgery was sterilized with three rounds of betadine followed by 70% isopropanol prior to making a 1 cm incision in the skin and in fascia above the quadriceps muscles. Using surgical scissors, a 3-4 mm defect was created in the mid-belly section of the quadriceps muscle by removing ⅓ of the quadriceps muscle. After removal of muscle, resulting tissue gap was filled with uniform amount (50 ul) of either polyethylene particulate or porcine derived ECM scaffold. The wound was then subsequently closed with 3-4 wound clips and the procedure was repeated on contralateral leg. After the surgery mice were placed under a heat lamp for 2-3 minutes to let them recover from anesthesia. Mice were then placed back in cage and were left on regular diet with enrichment until the end of the study. The protocol was approved by the NIH Clinical Center Animal Care and Use Committee under animal protocol number NIBIB 20-01.

### Flow Cytometry

After 3-, 7-, 21- and 42-days post injury, mice were ethically sacrificed followed my harvesting of injured muscle along with scaffolds. The dissected muscle was then finely diced and digested with digestive media (0.5 mg/mL Liberase™ (Sigma) and 0.2 mg/ml DNase I (Roche) in HEPES supplemented media) on a shaker at 100 rpm for 45 minutes and 37° C. The digested suspension was then filtered through 70 μm cell strainer and washed with 1×PBS and centrifuged at 350 g for 5 minutes at room temperature. The cell pellets were then soaked for 10 minutes in 5 mM EDTA in 1×PBS solution to reduce the cell clumping. After, 10 minutes of incubation, cells were then again washed with 1×PBS followed by centrifugation at 350 g for 5 minutes at 4° C. The pellets were then re-suspended in 200 μL viability dye solution (1:1000 dilution of Live/Dead Blue (Thermo Fisher) in PBS) for 20 minutes on ice followed by washing with wash buffer (1% BSA and 2 mM EDTA in 1×PBS). The cells were then stained with either myeloid panel or lymphoid panel antibodies (Tables 1 &2) followed by incubation at 4° C. for 30 min. After incubation the cells were washed thrice with wash buffer and analyzed on Cytek Aurora.

### Intracellular Staining

After surface staining, cells were fixed and permeabilized by utilizing True-Nuclear™ Transcription Factor kit (BioLegend) for intracellular staining of FoxP3 and HELIOS antibodies (Table 2) as per manufactured guidelines. Briefly, after last washing of surface staining, cells were resuspended in True-Nuclear™ 1× Fix Concentrate and incubated for 45 minutes at 4° C. After incubation the cells were centrifuged at 400×g for 10 minutes at 4° C. and resuspend in True-Nuclear™ 1× Perm Buffer. The cells were then again washed for one additional time with 1×Perm Buffer and were then treated with HELIOS and FoxP3 antibody cocktail (1:100 dilution of antibodies in True-Nuclear™ 1× Perm Buffer) followed by incubation at 4° C. for 45 minutes. After incubation cells were washed twice with True-Nuclear™ 1× Perm Buffer. After final wash, the cells were re-suspended in wash buffer and were analyzed on flow cytometer.

### Histopathology

Samples were fixed in 10% neutral buffered formalin for 48-72 hours prior to transfer to 70% ethanol. Samples were then dehydrated in graded ethanol steps through 70%, 80%, 95%, and 100% ethanol prior to clearing in xylene and embedding in paraffin wax. Quadriceps muscle groups were then cut in a transverse fashion to expose the center of the injury, which was then mounted face down in the paraffin mold. Five (5) to 7 m sections were then placed onto charged glass slides and baked overnight at 56° C. to dry. After rehydration, samples were stained with hematoxylin and eosin (H&E) through a 5-minute incubation in Harris Hematoxylin, followed by a wash in tap water and two dips in acid ethanol to destain, prior to rinsing in ethanol and dipping in Eosin Y. Samples were then washed in 95% ethanol, dehydrated, coverslipped and mounted with Permount. Picosirius red (PSR) staining occurred via manufacturer's instructions, after rehydration, samples were stained in PSR for 1 hour prior to washing in water and destaining with two dips in acetic acid then dehydrated and mounted in Permount. Slides were imaged on an EVOS microscope (Thermo).

### Immunohistochemistry

Samples were rehydrated, then incubated for 20 minutes in citrate antigen retrieval buffer prior to slowly cooling for 20 minutes on the benchtop. Endogenous peroxidases were quenched through a 5-minute incubation in 0.3% hydrogen peroxide in 1×PBS. Samples were stained using the VECTASTAIN® Elite ABC-HRP Kit (Rabbit, Vector Laboratories) as per manufacturer's instructions. Briefly, after washing in 1×PBS, samples were blocked in 2.5% normal goat serum for 1 hour. Samples were incubated in primary antibody diluted in blocking buffer for 1 hour. Rabbit monoclonal anti-CD103 (AbCam) and anti-E-Cadherin (AbCam) were diluted at a 1:100 dilution. Slides were washed 3 times in 1×PBS prior to incubation with biotinylated secondary antibody for 30 minutes. Slides were washed 3 times in 1×PBS then incubated for 30 minutes in the VECTASTAIN Elite ABC Reagent. Samples were washed 3 times in 1×PBS, and then incubated in ImmPACT® DAB EqV Peroxidase (HRP) Substrate (Vector Laboratories) for 1 minute and 30 seconds (CD103) or 50 seconds (E-Cadherin). Slides were washed in tap water, then counterstained for 5 minutes in Harris Hematoxylin (Sigma), prior to rinsing in tap water, destaining in acid ethanol, dehydration and mounting in Permount.

### RNA Isolation and RT-PCR

Quadriceps muscle group was dissected from mice at 7 days post-injury and homogenized in 2 ml 1×PBS with a mechanical homogenizer at 5000 rpm for 30 seconds. Five hundred (500) microliters of the resulting homogenate was transferred to an Eppendorf tube containing 500 ul of TRI Reagent Solution (Sigma Aldrich). Samples were vortexed and then stored at −80C until RNA isolation. After thawing, 200 ul of chloroform (Sigma Aldrich) was added to each sample and vortexed before being allowed to separate for 5 minutes at room temperature, followed by centrifugation for 15 minutes at 8000×g and 4° C. Aqueous phase was combined with an equal volume of 70% ethanol and vortexed. The resulting sample was passed through an RNeasy Mini Prep spin column (Qiagen), and then washed as per manufacturer's instructions with 1 wash of RW1 Buffer, and 2 washes of RPE Buffer. The membrane was dried and RNA was eluted into 30 ul of RNAse-free water. RNA concentrations were determined by NanoDrop and quality control was performed to move forward with samples with and A260/A280>2. Samples were diluted to 100 ng/ul concentration, and 11 ul were added to a SuperScript Reverse Transcriptase IV reaction following manufacturer's instructions with Random Hexamers as primers (ThermoFisher Scientfic). Two (2) ul of the resulting cDNA was added alongside 10 ul of TaqMan Fast Advanced Master Mix, 7 ul of nuclease-free water, and 1 ul of FAM-MGB primer/probe: Gusb, Xcl1 (Table 3).

Protein Isolation from Mouse Tissue and Cytokine Analysis

Muscle and lymph node samples were flash frozen in liquid nitrogen or an ethanol-dry ice slurry immediately after dissection and stored until processing. Frozen muscle samples were added to 2 ml ice cold 1×PBS with protease inhibitors (ThermoFisher Scientific) and diced with a pair of scissors. Samples were homogenized for 45 seconds using a mechanical homogenizer at 5000-6000 rpm while on ice. Subsequently, 2.5 ml more ice cold 1×PBS with protease inhibitors were added along with 50 ul of 10% Triton-X100 then mixed vigorously and left on ice for 5 minutes prior to aliquoting and snap freezing in liquid nitrogen and stored until use. Day of use, samples were thawed and centrifuged at 10,000×g for 10 minutes to pellet debris. Protein concentration was determined via the Pierce BCA Protein Assay Kit (Thermo Scientific) at a 1:1 dilution with lysis buffer. Two hundred (200) micrograms of protein were loaded onto a Proteome Profiler™ Array, Mouse XL Cytokine Array Kit and assayed per manufacturer's instructions. Blots were imaged on a BioRad ChemiDoc with a 30 second exposure.

### Enzyme-Linked Immunosorbent Assay

The XCL-1 measurement in mouse blood plasma samples were performed by using Mouse XCL-1 SimpleStep ELISA kit (Abcam). The assay was performed as per manufacturer guidelines. Briefly, 50 μL of 1:1 diluted mouse blood plasma sample or protein lysate with blocking buffer were added to appropriate wells of precoated 96 well plate. The samples were then treated with 50 μL of antibody cocktail followed by incubation for 1 hour at room temperature. After incubation, the mixture in wells were aspirated and wells were washed three times with wash buffer. After final wash, 100 μL of TMB development solution was added to each well and plate was incubated for 10 minutes. After incubation, 100p L of stop solution were added in each well followed by reading OD at 450 nm.

### Statistics and Data Analysis

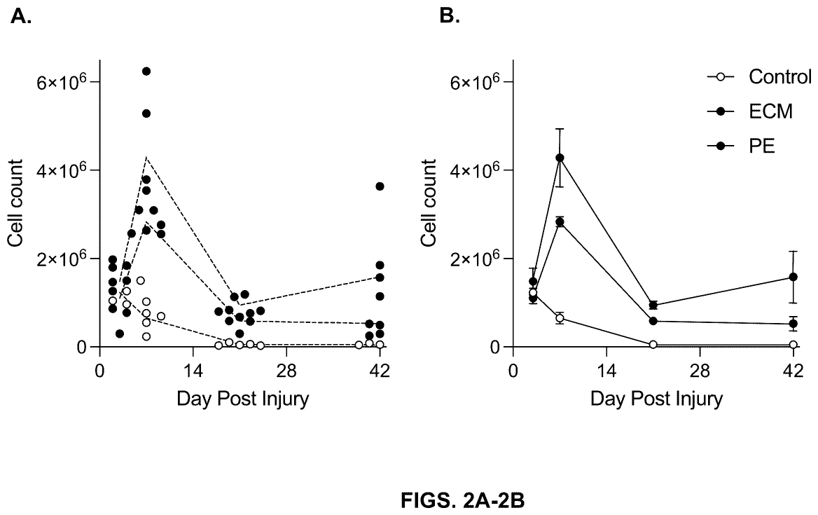

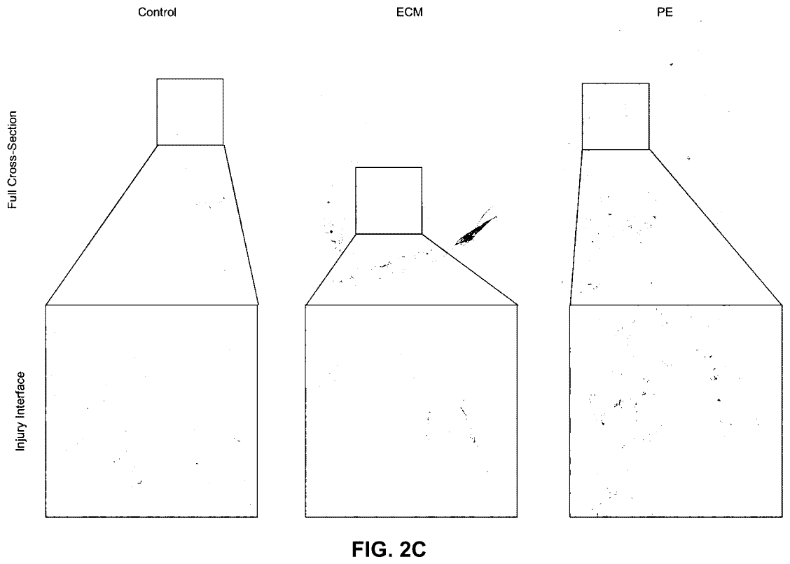

Flow cytometry data were unmixed using stated single spectra controls (Supplemental Tables 1, 2) using SpectroFlo Software (Cytek Biosciences). Resulting unmixed data were exported to .fcs prior to analysis on FlowJo (Supplemental FIGS. 2,16). Dimensionality reduction algorithms were fund through FlowJo plugins. t-stochastic neighbor embedding (t-SNE) was run at the following parameters: learning configuration—opt-SNE, iterations—2000, perplexity—30, KNN algorithm—exact (vantage point tree), gradient algorithm—Barnes-Hut. Uniform manifold projection was run at the following parameters: Euclidean, nearest neighbors—15, minimum distance—0.5, Number of components—2. FlowSOM 3.0.18 was run at the following parameters: Number of meta clusters—30. Clustering was run on singlet live immune cells using all parameters excluding LIVE/DEAD Blue. Resulting data from manual gating were analyzed in GraphPad Prism v9 and R 4.1.2. Immunohistochemistry of E-Cadherin was quantified through auto-white balance in Fiji (ImageJ) followed by Color Deconvolution to isolate the DAB channel. Areas of interest were manually outlined then measured and transformed into optical density (OD) readings by taking the log (max intensity/Mean intensity). Each replicate represents quantification of a section from a different animal. Resulting OD values were plotted in GraphPad Prism v9 for data display and analysis. Chemilumiescent proteome profiler blots were quantified by pixel intensity via MatLab (version R2022a) using the Protein Array Tool version 2.0.0.1 and normalized to background prior to being displayed as a fold change over uninjured control muscle tissue in R 4.1.2. Technical duplicates of RT-PCR Ct values were averaged after subtracting housekeeping gene (Gusb) then the average ΔCt of uninjured control was subtracted from all ΔCt values prior to transforming to display fold change as 2(−^{ΔΔ}Ct).

Statistical tests used are stated in figure captions.

### Example 1. Characterization of Immune Cell Response Following Injury

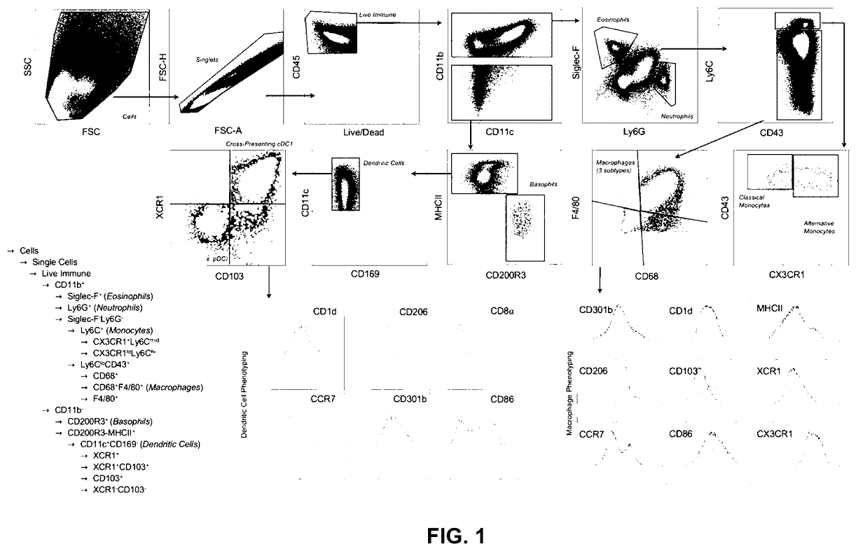

To evaluate immune responses to engineered materials in trauma and tissue regeneration, bilateral volumetric muscle loss surgeries were conducted followed by material implantation. After a 3 mm defect was created in the quadriceps muscle group, the resulting void was backfilled with either a control (saline), hydrated decellularized extracellular matrix (ECM) powder, or polyethylene powder (PE). The ECM provides an example of a pro-regenerative material, and the PE is an example of a pro-fibrotic material. A 22-color flow cytometry panel was used to interrogate the myeloid immune response to early stages of material response and trauma healing, including markers for granulocytes, macrophages, dendritic cells, and phenotyping their activation (FIG. 1). Material treatment yielded an increase in cellular infiltrate that peaked at 7 days post injury and maintained at half of the peak infiltration level out to 42 days post-injury. Saline-treated controls peaked cellular infiltration early, by 3 days post-injury (FIG. 2A-2C). Structurally, the ECM material degraded over time but was still present by 41 days post-injury as noted by picrosirius red staining for collagen. Cells that had granular cytoplasms and segmented nuclei associated with neutrophils were found in coronas around the PE particles, which was subsequently replaced by a fibrotic capsule. Cellular infiltration into the material area correlated with overall cell counts observed during flow cytometric analyses (FIG. 2A-2C).

A. Implanted Materials Recruit a Diverse Innate Immune Compartment with a Granulocyte Shift from Eosinophil-Dominant to Neutrophil-Dominant Repertoires with Fibrotic Materials

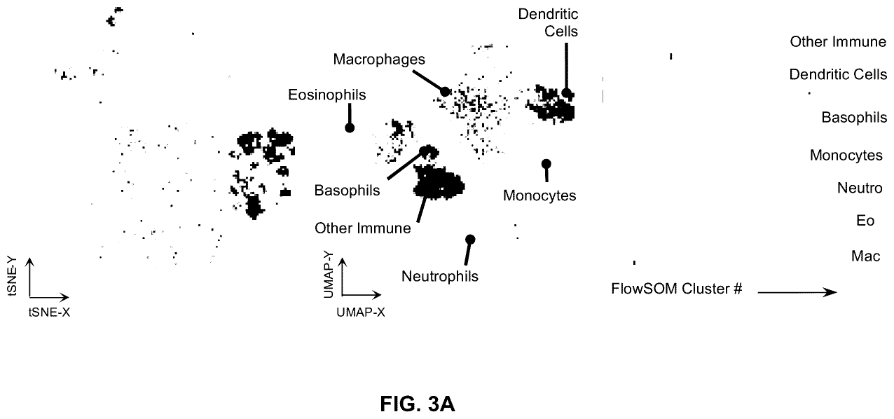

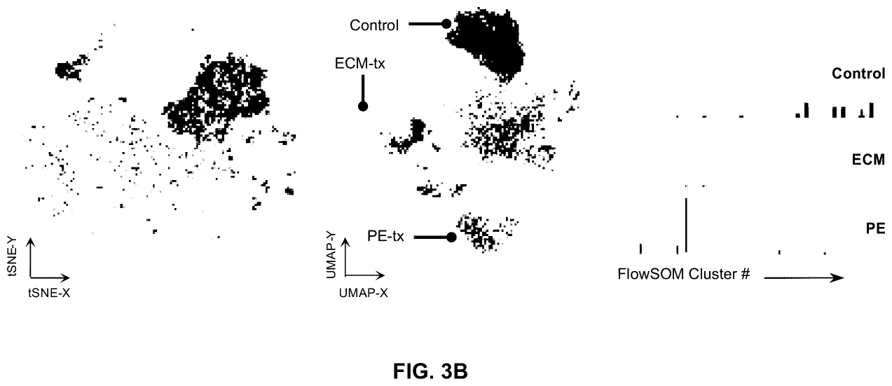

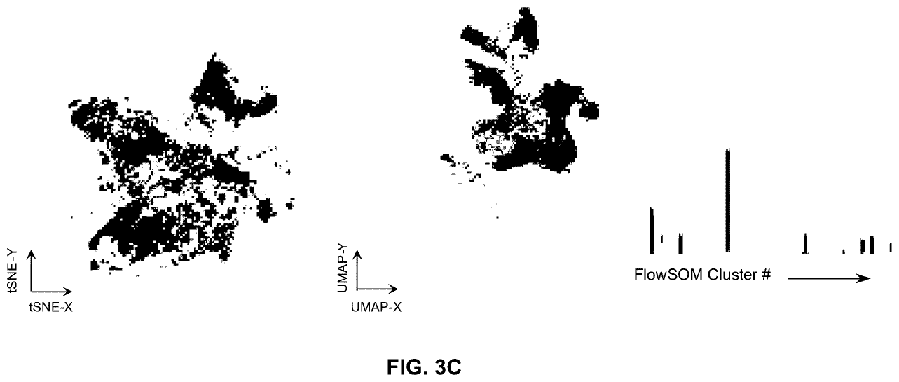

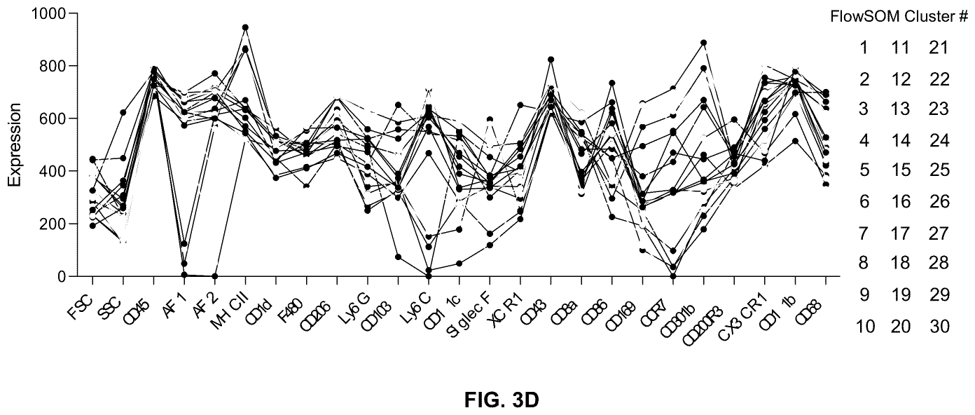

Flow cytometry showed that a varied set of innate immune cells was recruited to the injury microenvironment by 7 days post-injury which was dependent upon material treatment and visualized via dimensionality reduction (FIGS. 3A-3D). Granulocytes, such as neutrophils, basophils, and eosinophils, as well as mature macrophages and immature monocyte-like myeloid cells, dendritic cells, and other immune cells (CD45+Lin−) (FIG. 3A), were identified through manual gating. A comparison of the response to different materials revealed a divergence in the immune repertoire by 7 days post-injury (FIG. 3B). Different sub-populations of macrophages and dendritic cells were identified, and the presence of granulocyte and monocyte-like cell populations confirmed using FlowSOM, a self-organizing map (SOM) algorithm for generation of clusters based on the expression of markers detected via flow cytometry (FIGS. 3C-3D).

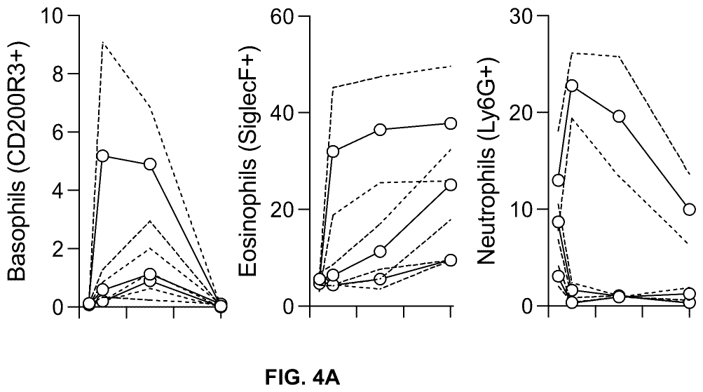

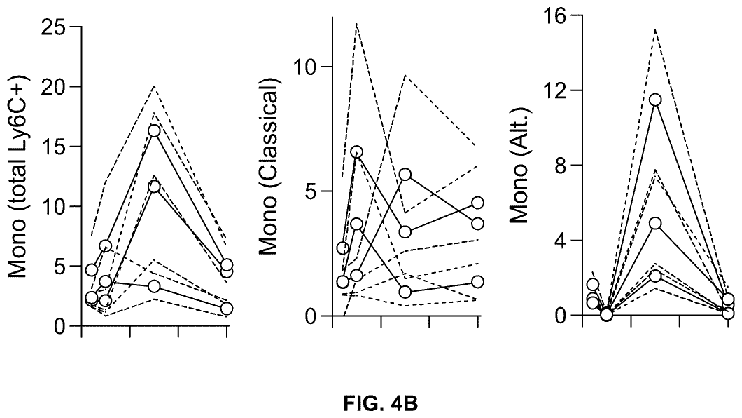

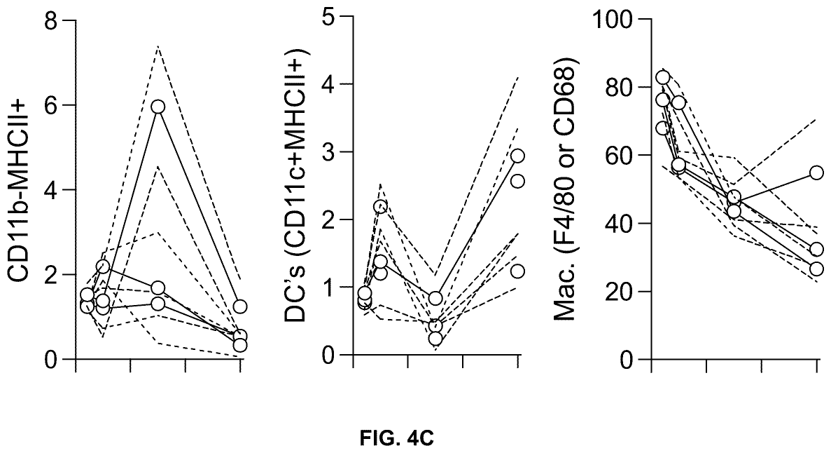

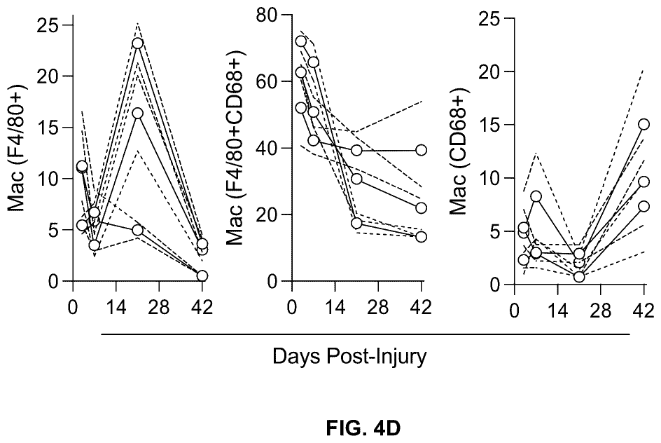

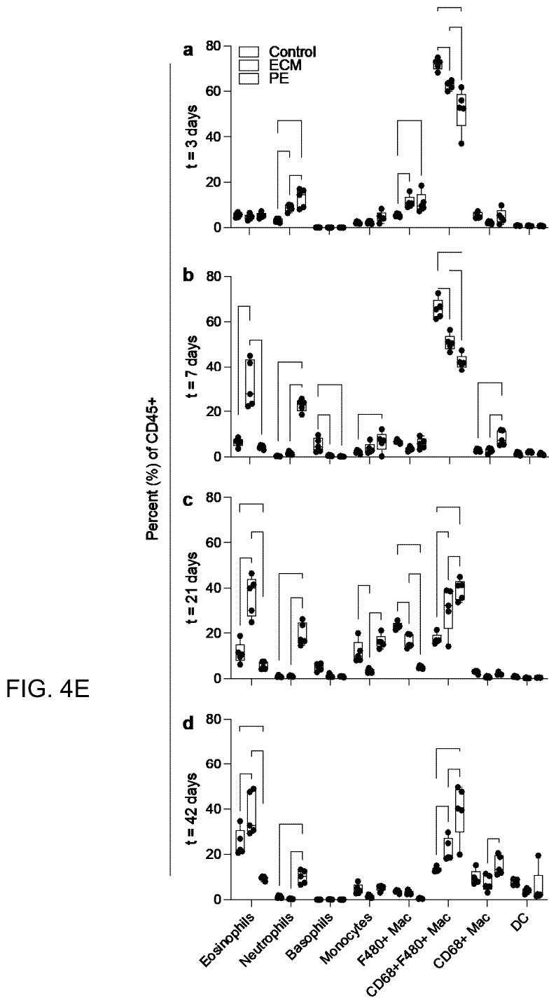

As previously described, a high prevalence of macrophages (F4/80+CD68+) that persist throughout the course of injury recovery (FIGS. 4A-4E) was observed. Further, a preferential recruitment of neutrophils (Ly6G+) to pro-fibrotic PE-treated muscle injury was seen. Pro-regenerative materials produce an eosinophil-dominant granulocytic compartment that are more autofluorescent than neutrophils, possibly due to granule formation or phagocytosis of extracellular matrix material. CD200R3+ Basophils were preferentially recruited to untreated control injuries and peaked between 7 to 21 days post injury whereas eosinophils in ECM-treated injury persist from 7 through 42 days post-injury, and neutrophils in PE-treated injury peak by 7 days post-injury and slowly decline by 42 days post-injury while still maintaining a large proportion of overall immune cells in the microenvironment. Control and ECM-treated injuries both recruited neutrophils early on but they were cleared by 7 days post-injury. PE-treated injuries recruited higher levels of monocytes in comparison to other treatments, with a preference to CX3CR1+ cells that may represent activation of a pathogenic type 2 immune response that promotes fibrosis as the neutrophilic inflammation begins to subside. Dendritic cells were present and persist in a low proportion (<4% of total CD45+ cells) throughout the time course of response to injury and material implantation. Macrophages peaked early and began to decrease in proportion with time, with a shift from CD11b+F4/80+CD68+ cells to mostly CD11b+CD68+F4/80− cells by 42 days post-injury.

### B. CD103+XCR1+ Dendritic Cells are Enriched by Pro-Regenerative Scaffolds

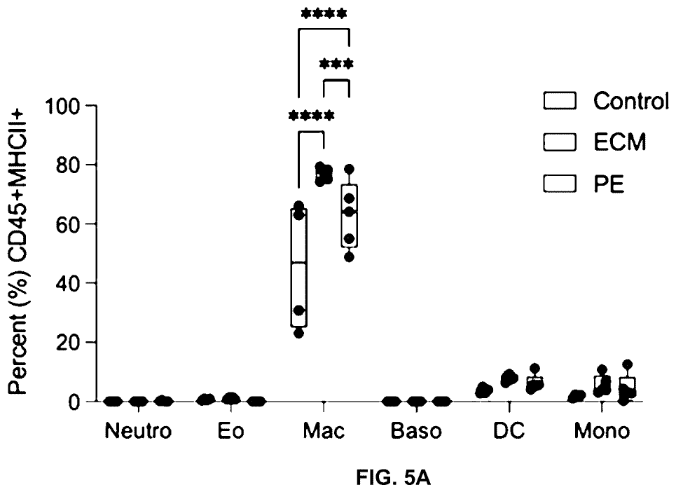

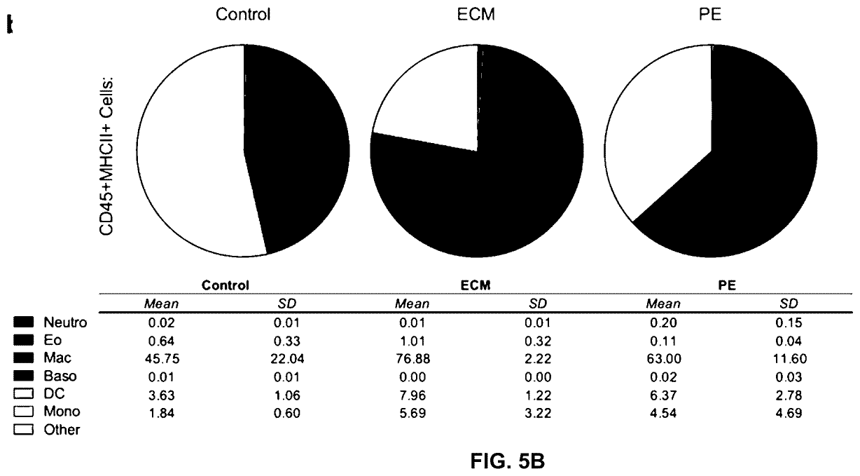

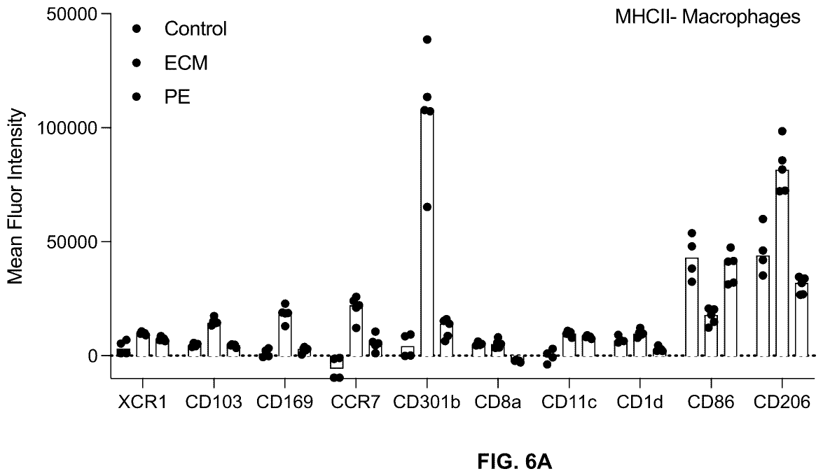

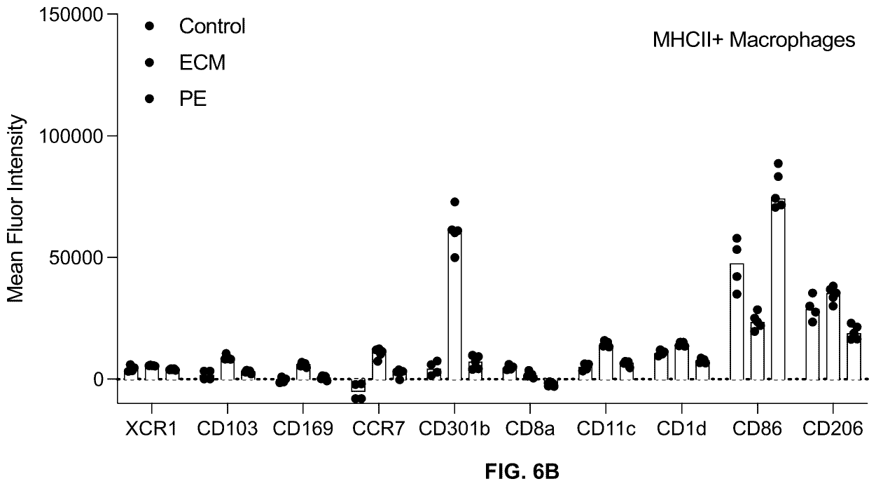

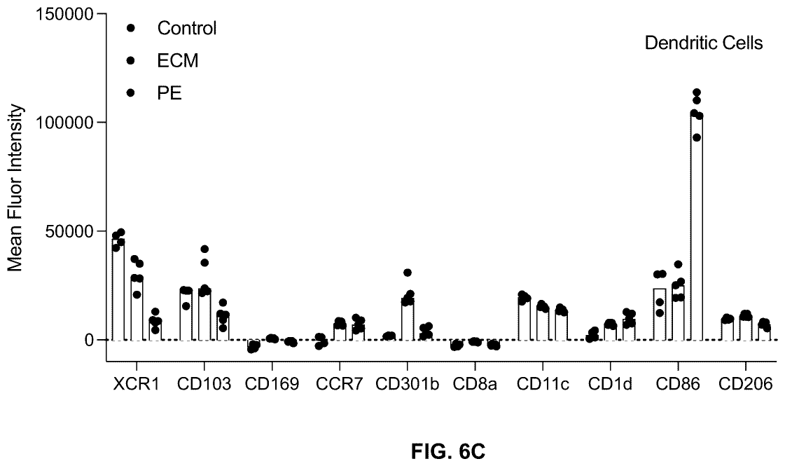

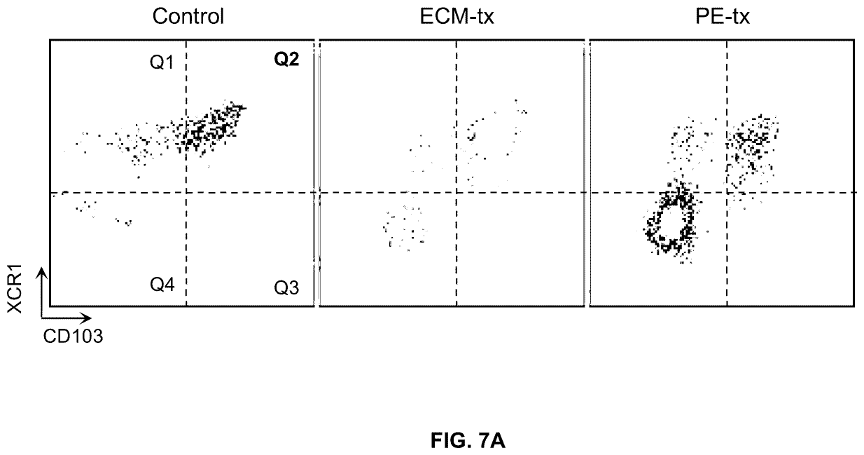

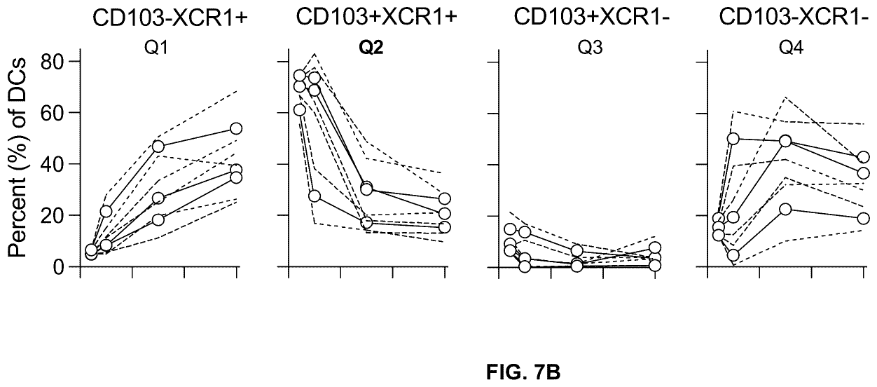

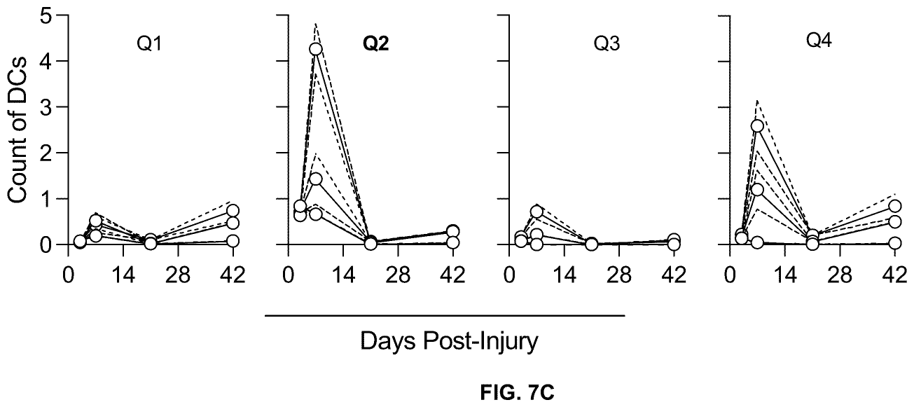

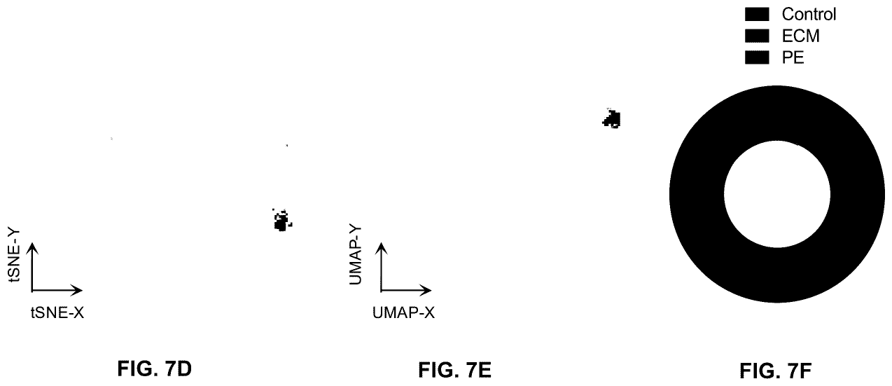

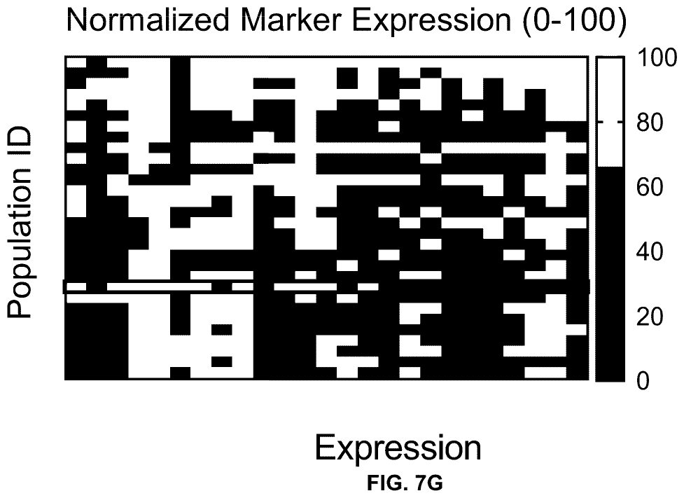

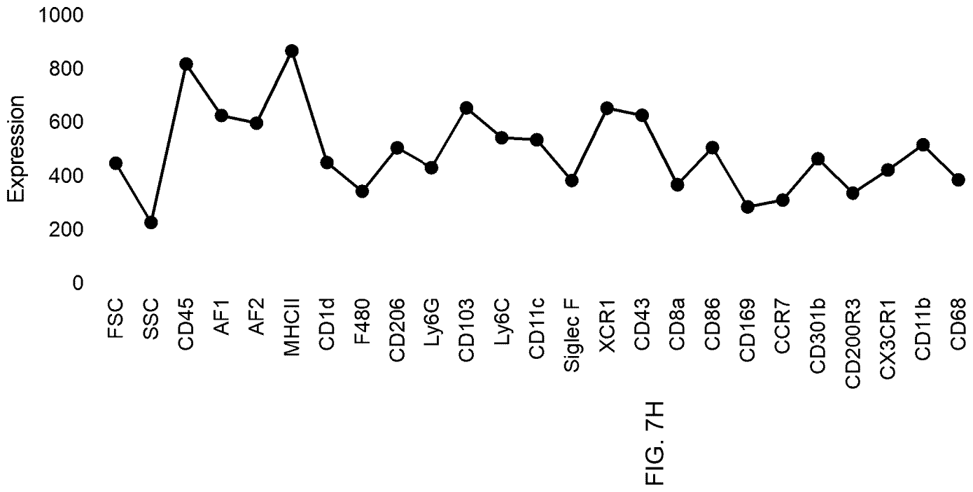

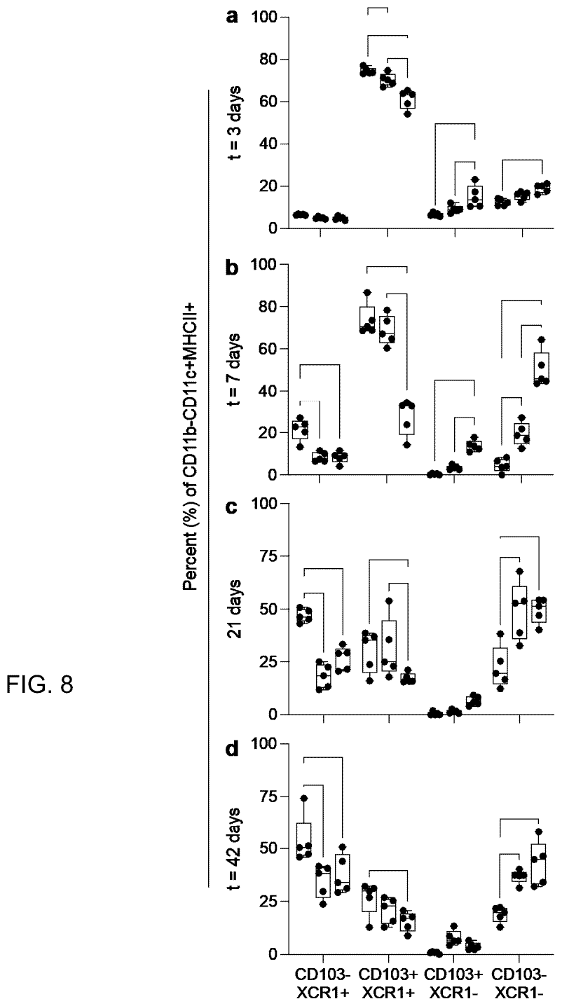

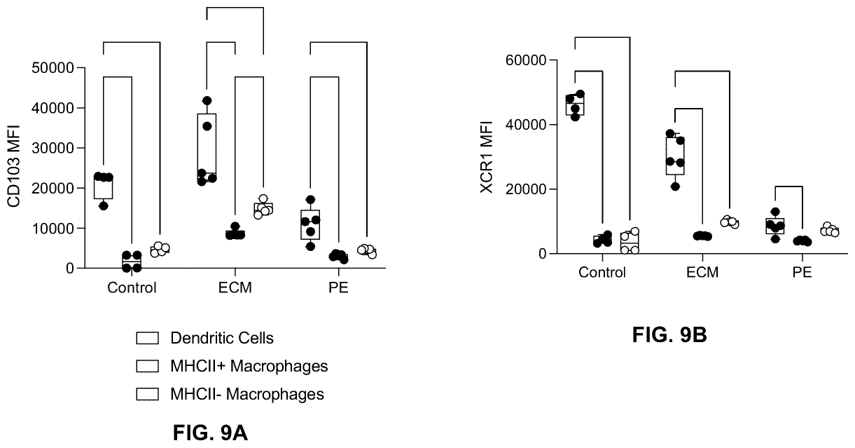

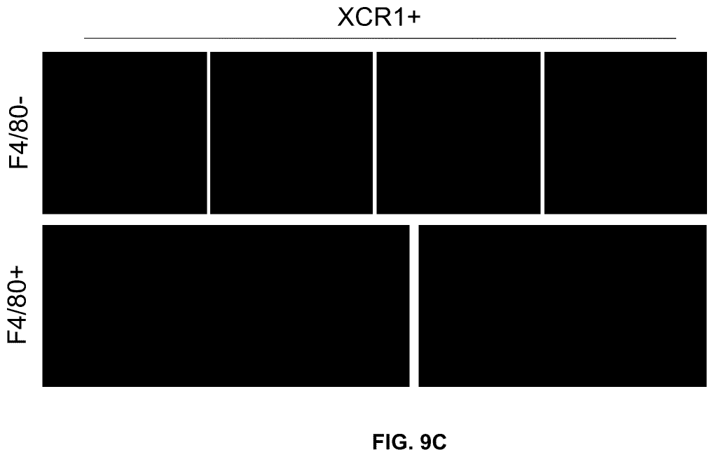

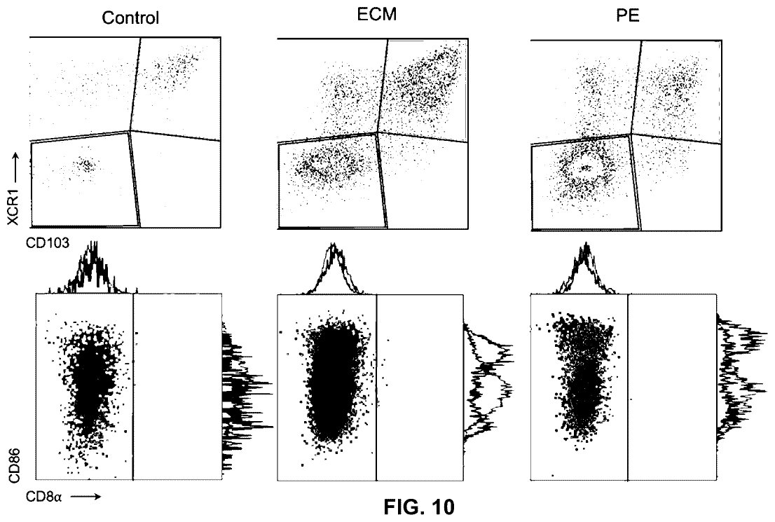

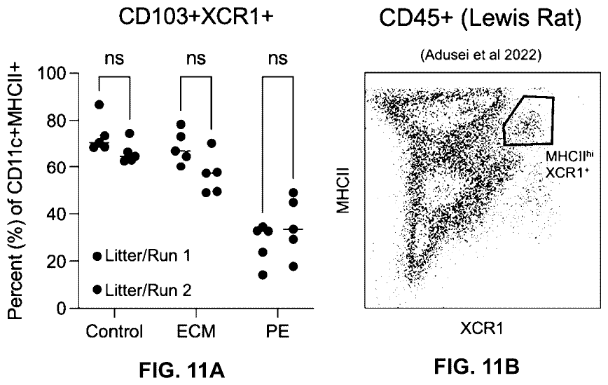

As previous studies have implicated adaptive immunity in the biomaterial integration and regeneration/fibrosis processes, the phenotype of antigen presenting cells was further investigated. The majority of MHCII+ immune (CD45+) cells in the wound microenvironment were F4/80+ macrophages (FIGS. 5A & 5B). It was found that macrophages in ECM-tx muscle injury had higher levels of CD206, CD301b, and CD169 expression suggesting local proliferation of tissue-resident cells with a type-2 polarization which is a hallmark of Th2 driven inflammation (FIGS. 6A-6C). While only representing 1-2% of the total CD45+ immune cell infiltrate, the second most common APC in the wound space were dendritic cells (CD11c+CD11b^{lo/neg}) Identification of cross-presentation capable dendritic cells was determined by the expression of XCR1, a chemokine receptor, and CD103 on CD11b⁻CD11c⁺MHCII^{hi} dendritic cells (FIGS. 7A-7H, FIG. 8). CD11b⁺F4/80⁺ macrophages also expressed low levels of CD103 and XCR1, but significantly less than the CD11c+CD11b^{lo} dendritic cells (FIG. 8, FIGS. 9A-9C). Type 1 conventional dendritic cells (cDC1s) were enriched by pro-regenerative scaffolds whereas pro-fibrotic scaffolds recruited mainly double negative cells in a pattern that persisted to 42 days post-injury (FIGS. 7A & 7B). cDC1s expressed intermediate levels of the co-stimulatory molecule CD86, whereas the double negative cells showed a bimodal distribution with a sub-population of CD86^{hi} dendritic cells that may correlate with plasmacytoid DCs (pDCs) (FIG. 10). Neither population expressed CD8α in the muscle tissue. Double negative and XCR1 single positive cells increased in proportion for all groups with time (FIGS. 7B & 7C), but XCR1⁺CD103⁺ conventional dendritic cells are peak in proportion by 3 to 7 days post-injury and by count at 7 days post-injury (FIGS. 7B & 7C). There were very few CD103 single positive dendritic cells in the wound space throughout the course of injury recovery (FIGS. 7B & 7C). When evaluating cell populations through dimensionality reduction and hierarchical clustering algorithms, the FlowSOM algorithm identified this cell type as a unique cluster, and it was mapped to an island with both t-SNE and UMAP visualizations (FIGS. 7D & 7E). When FlowSOM was applied to the three treatment groups within the same clustering population, over 70% of the cells within the cDC1 cluster were from the ECM-treated injury whereas only 15% and 6% were from PE treatment and Control injuries, respectively (FIGS. 7F-7H). Identification of these cells was confirmed in two C57BL/6 mouse litters as well as in the muscle injury of a Lewis Rat suggesting this is repeatable and applicable to multiple species within the Muridae family (FIGS. 11A-11B).

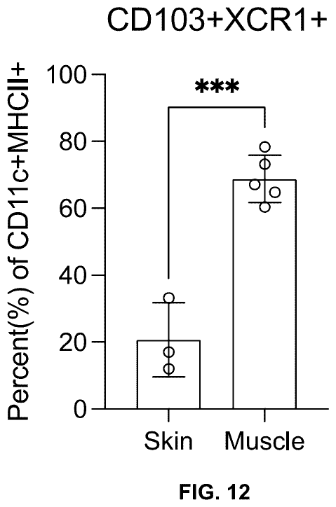

At 7 days post-injury when CD103+XCR1⁺dendritic cells peaked in the muscle tissue, these dendritic cells were detected in the overlaying skin incision site of ECM-treated muscle injury, but in a significantly lower fraction, possibly because the skin wound was largely closed by this time (FIG. 12).

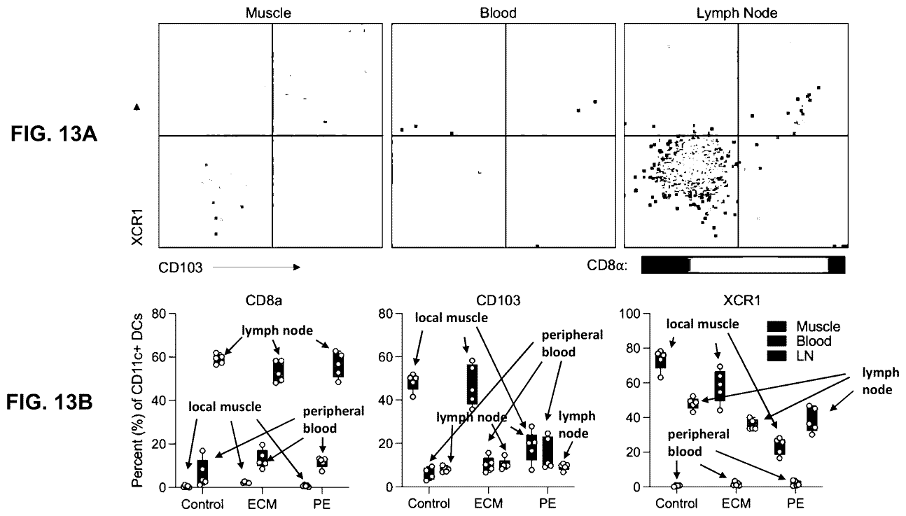

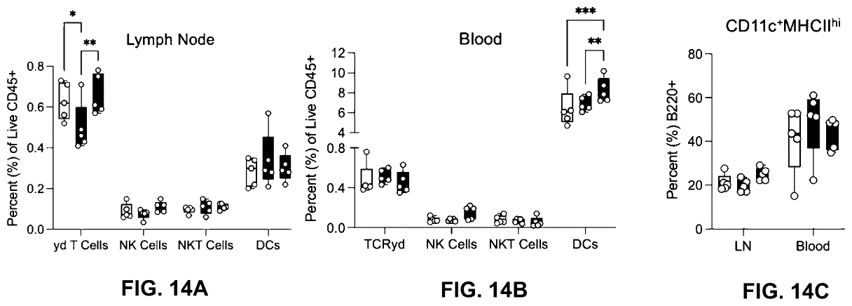

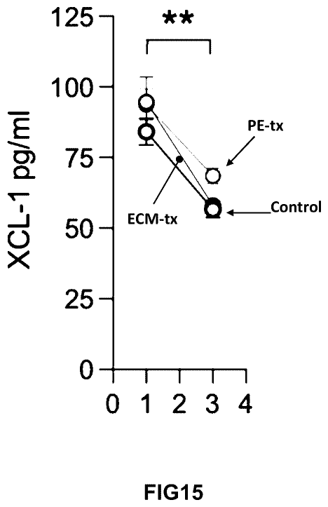

Outside of the local muscle tissue dendritic cells were detected in the blood and lymph nodes of injured mice. These DCs (CD11b^{-/lo}CD11c⁺MHCII^{hi}) had differing expression of markers associated with antigen cross-presentation capacity (XCR1, CD103, and CD8a) depending upon their location (FIGS. 13A-13B). As mentioned, in muscle, cells expressed high levels of both XCR1 and CD103, with no expression of CD8a. In the lymph node, these cells expressed all three markers, XCR1, CD103 and CD8a, whereas in the blood most cells were XCR1⁻CD103⁻CD8a⁻. Interestingly, there was a larger proportion of CD11c⁺MHCII^{hi} DCs in the blood of PE-treated mice, and most of these cells were B220/CD45R⁺ suggesting a potential preference towards circulating pDC recruitment for pro-fibrotic material implants (FIGS. 14A-14C). In the lymph node there was no significant difference in the proportion of dendritic cells, though PE-treated mice did have a higher proportion of TCRγδ+ T cells agreeing with previous literature on the role of these cells in fibrotic disease (FIG. 14A). Regarding the chemokine that binds XCR1, there was no significant difference between treatment groups in XCL-1 levels in the blood at 7- and 21-days post injury though in all treatment groups there was a significantly higher amount of XCL-1 in the blood earlier in response to wounding (FIG. 15).

### C. Phenotype and Recruitment of CD103+XCR1+ Dendritic Cells are Dependent on Adaptive Immune Activity

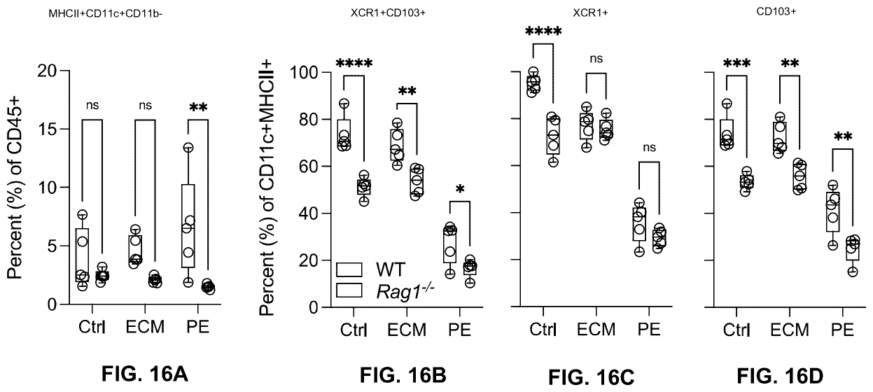

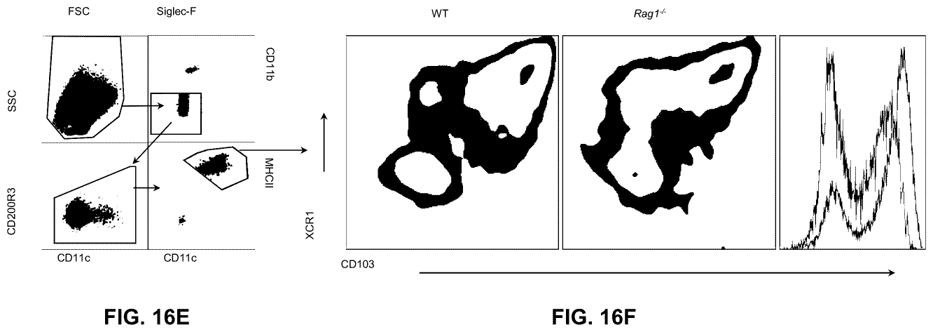

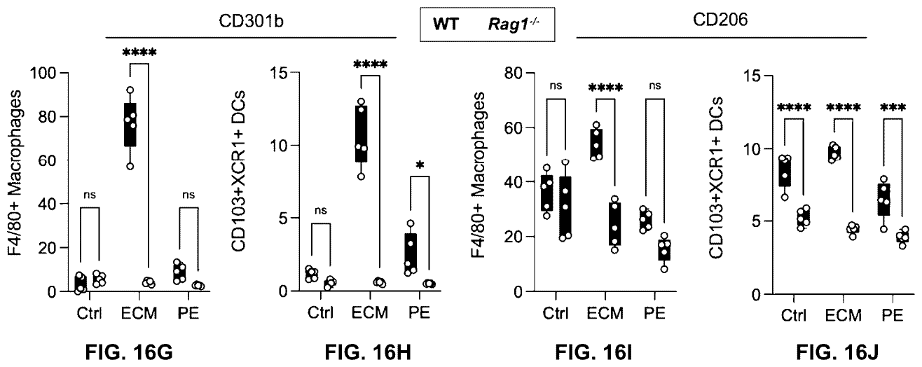

As dendritic cells are capable of direct communication with adaptive immune cells such as T cells and B cells through antigen presentation, the inverse relationship and role of adaptive immunity on recruitment and activation of these cells within the scaffold microenvironment was investigated. In RAG-deficient mice (Rag1^{-/-}), which lack T cells and B cells, there were similar proportions of dendritic cells recruited to the overall environment, trending fewer in Rag1^{-/-}, but significantly fewer XCR1+CD103+ cDC1s (FIGS. 16A-16F). When evaluating their recruitment based on expression of XCR1 or CD103, the loss of CD103 expression was the main contributor to the loss of these cDC1s.

As with macrophages, in wild type mice Dendritic Cells had high levels of CD301b and CD206 in the presence of ECM scaffolds (FIGS. 16G & 16H), with CD103+ DCs expressing the highest levels of CD206 in comparison to other DC subtypes (FIGS. 16I & J). ECM scaffold-mediated type-2 upregulation was lost for both CD301b and CD206 (FIGS. 16G-16J) in Rag1^{-/-} mice when compared to wild type mice.

### Example 2. Examination of T Cell Involvement in Material Response

### A. Early T Cell Activation Followed by Induction of Regulatory CD8+ T Cells is Enhanced by Pro-Regenerative Scaffold Treatment

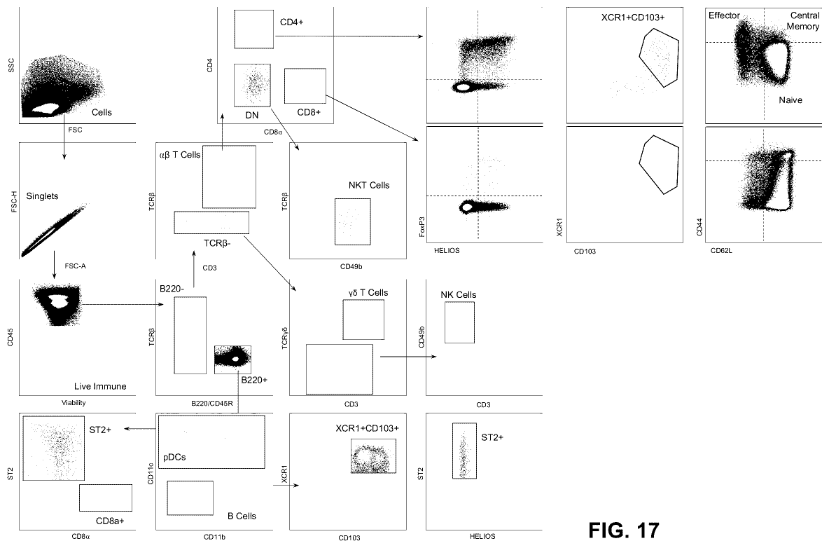

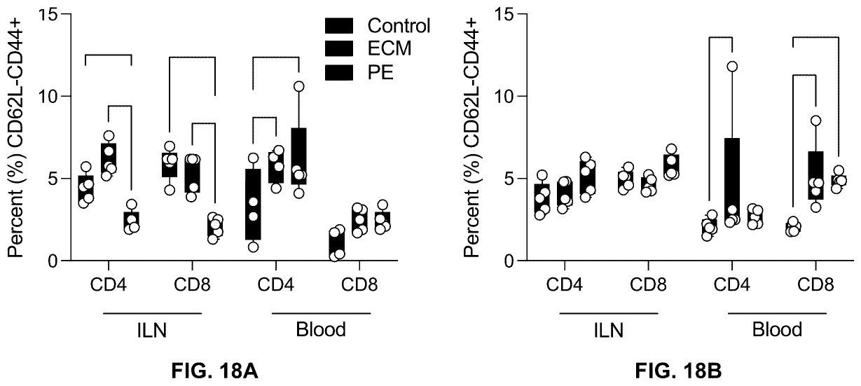

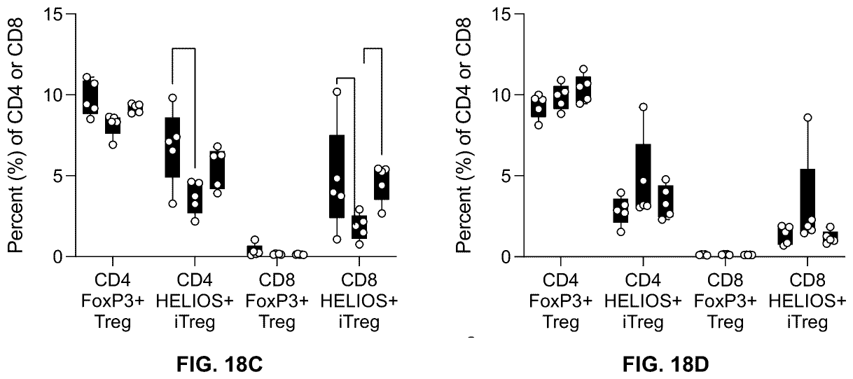

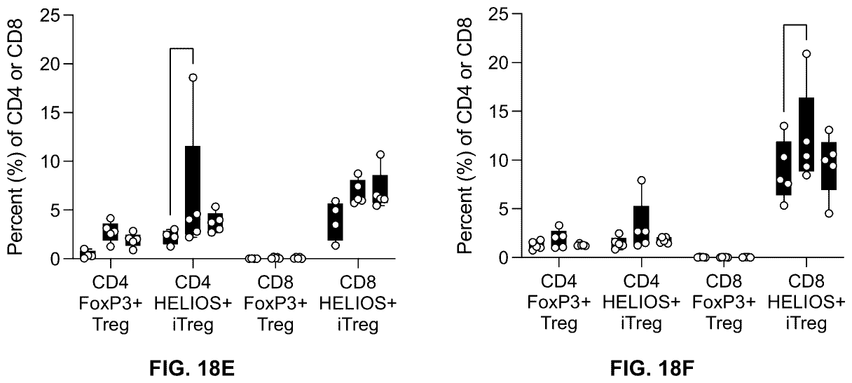

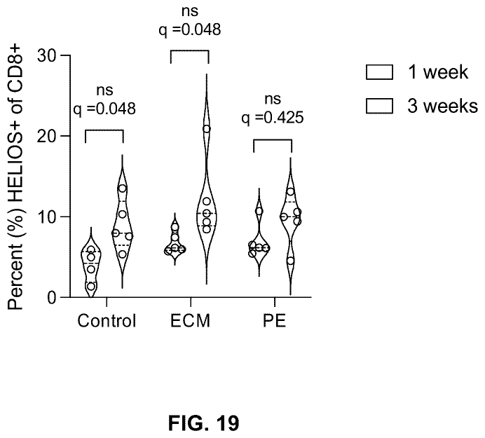

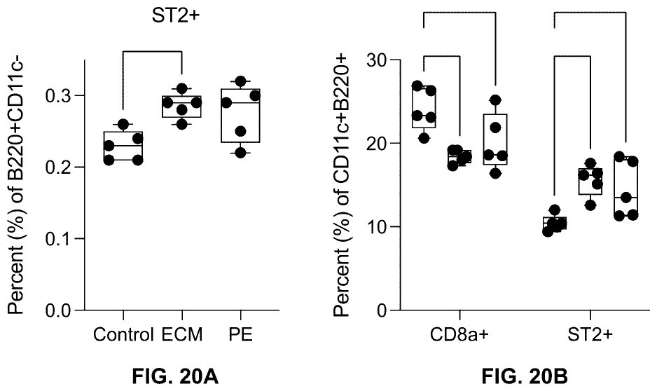

A 19-color flow cytometry panel was developed to evaluate lymphoid cell behavior in the blood and draining lymph node (FIG. 17). At 7 days post-injury there was an increased prevalence of CD44⁺CD62L⁻ CD4 and CD8 T cells in the draining lymph node in ECM-treated injury compared to PE-treated injury. Active CD4+ T cells in the blood were enriched by ECM treatment at 7 days post-injury correlating with previous work showing a peak in IL-4 expression in the draining lymph node at this time. This correlated with lower proportions of HELIOS+ regulatory CD4 and CD8 T cells in draining lymph node at 7 days post-injury. In the peripheral blood, there was a slightly higher prevalence of CD4+HELIOS+ iTregs in comparison to control injury, which was more prevalent at 21 days post-injury. All treatments were trending to higher proportions of HELIOS+CD8 T cells at 21 days post-injury in comparison to 7 days post-injury, though not significant after corrections for multiple comparisons (FIGS. 18A-18F, FIG. 19). In addition to these regulatory cells, we identified a higher proportion of ST2+ regulatory B cells in ECM-tx and ST2+ pDCs in both ECM-tx and PE-tx injuries when compared to control. Whether or not these cells functionally serve in a regulatory or pathogenic role is uncertain. (FIGS. 20A & 20B).

### B. CD103+XCR1+ Adaptive Immune Cells are Induced by Trauma and Modified by Material Treatment

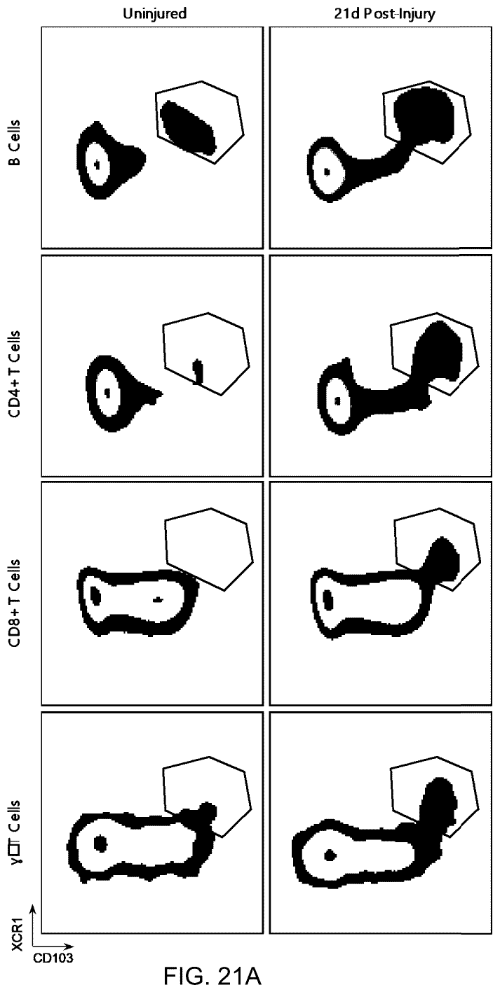

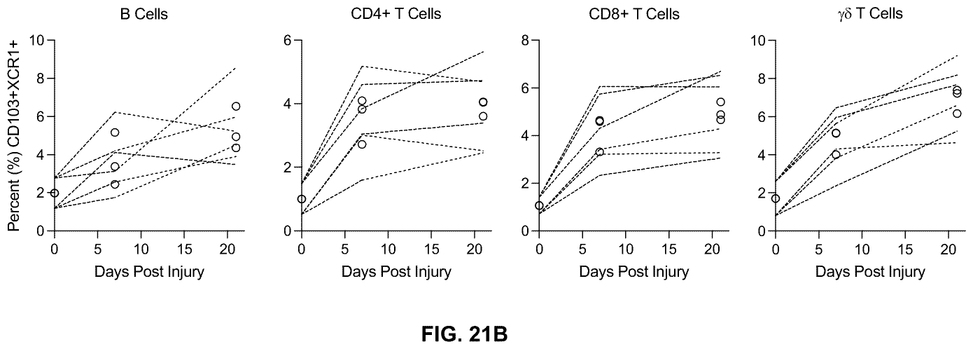

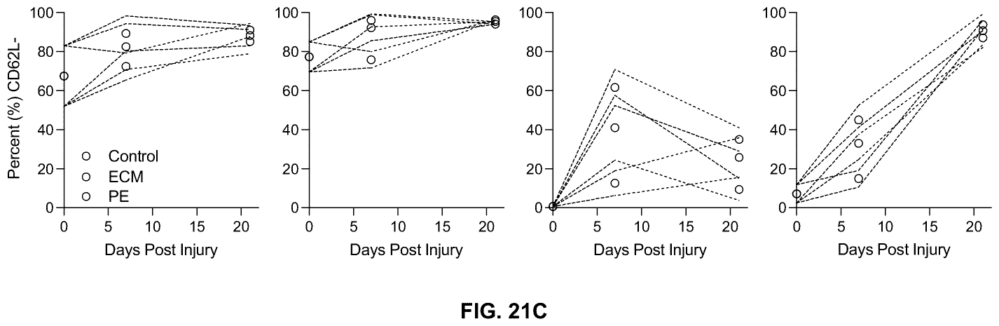

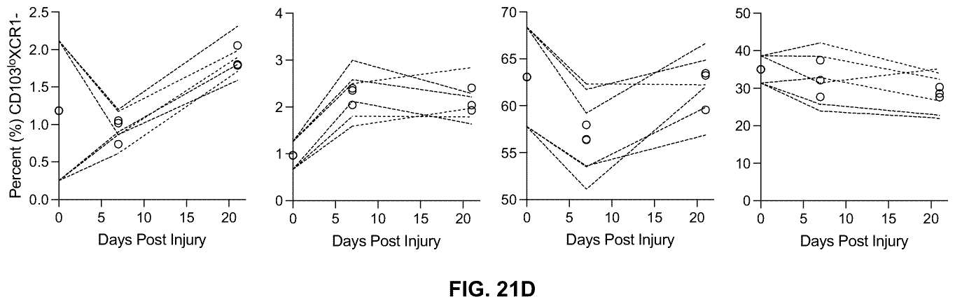

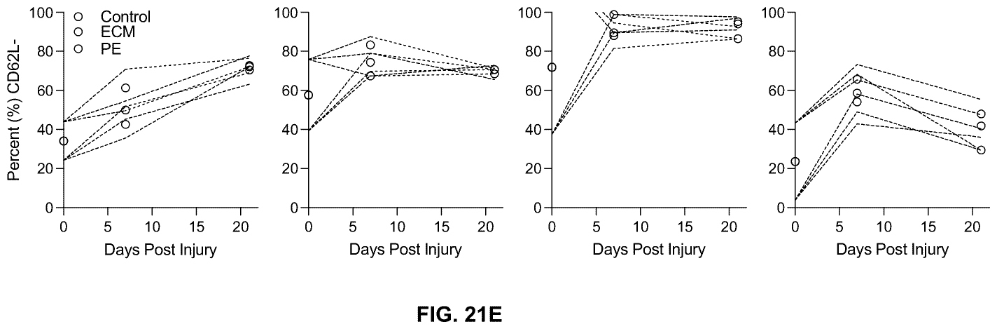

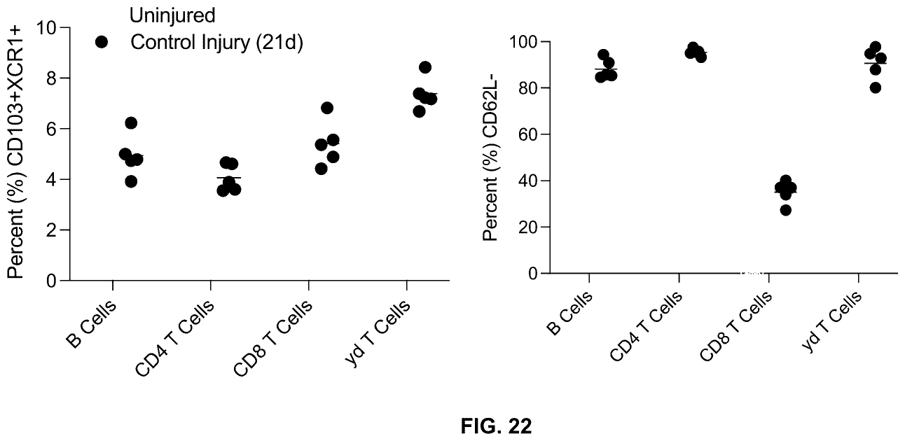

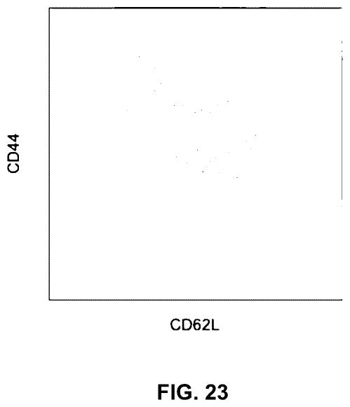

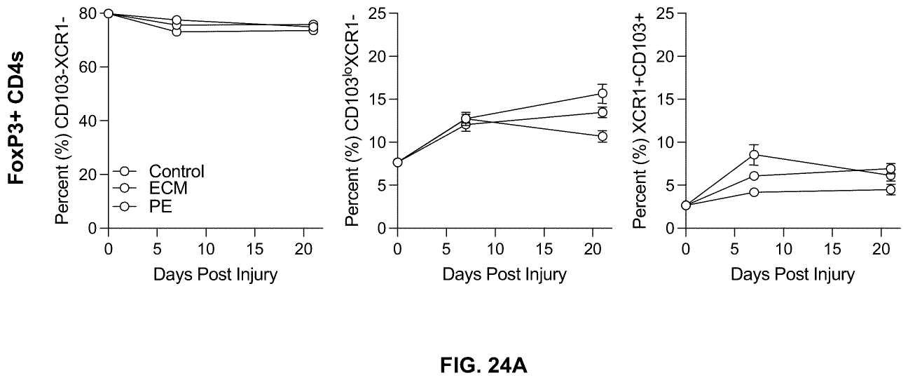

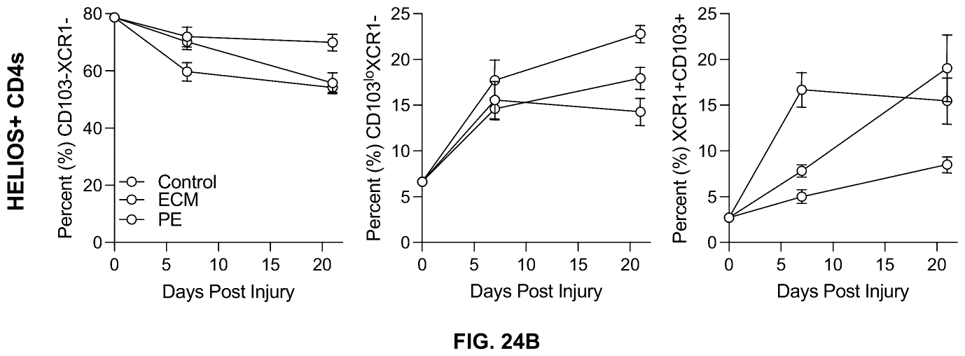

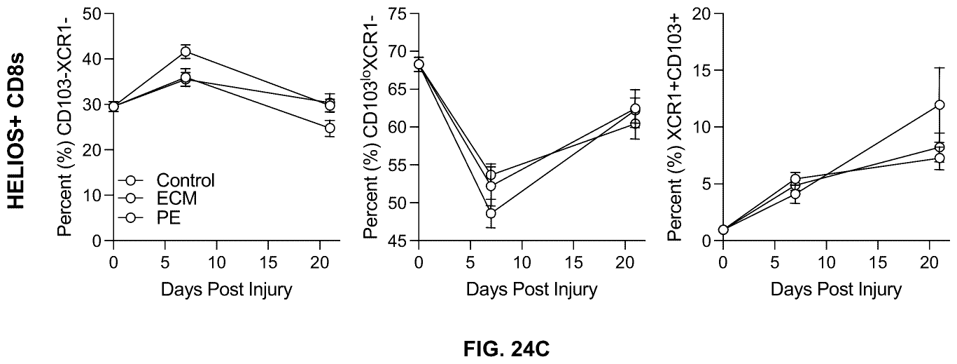

In addition to these regulatory T cells, a sub-population of CD103+XCR1+ adaptive immune cells was found that was induced by trauma (FIG. 21A, FIG. 22). This population was present for B cells, CD4+ T cells, CD8+ T cells, and γδ T Cells and increased with time (FIG. 21B). In B Cells and CD4+ T cells, most of this population was CD62L⁻ even without injury, and for CD8+ and γδ T Cells activation increased with time, with CD8s reaching their peak by 7 days post-injury and γδ T Cells increasing through 21 days post injury (FIG. 21C). In addition to the CD103+XCR1+ population, there was also a CD103^{lo}XCR1− population that was most prominent in CD8+ T cells and γδ T Cells (FIGS. 21A & 21D). Activation of these cells, as determined by the loss of CD62L expression, peaked by 7 days post injury (FIG. 21E). Interestingly, these cells were all CD44^{lo} in both blood and draining lymph node, possibly suggesting an antigen-independent activation though more work would be needed to test this hypothesis (FIG. 23). As there were multiple active adaptive immune cells in sterile injury, these CD103 and XCR positive adaptive immune cells were evaluated to determine if they behaved in a regulatory manner. It was found that the proportion of HELIOS+ iTregs that were CD103+XCR1+ increased over time, more so than FoxP3+HELIOS− Tregs (FIGS. 24A-24C). This pattern was true for both CD4+ iTregs as well as CD8+ iTregs.

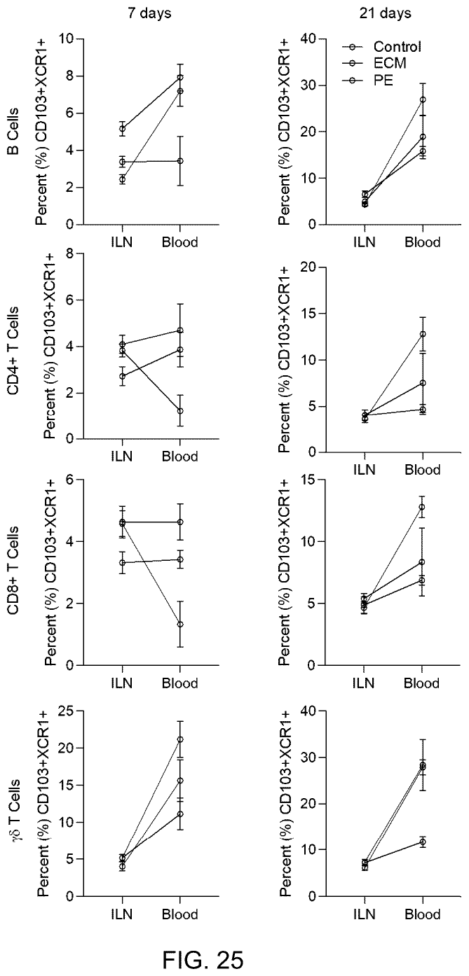

When comparing the draining lymph node and the peripheral blood, a higher proportion of CD103+XCR1+ adaptive immune cells in circulation was observed (FIG. 25). For all treatment groups, B cells had the highest proportion of CD103+XCR1+ cells at 21 days post-injury, followed by γδ T Cells, except for ECM-treated mice which had a lower proportion of these cells in circulation. Additionally, at 7 days post-injury when there was a peak in CD103+XCR1⁺dendritic cell recruitment, a strong enrichment of CD103+XCR1+ T cells was observed in the wound space in comparison to the blood and draining lymph node, suggesting there was a preferential recruitment of these cells to the injury space.

### C. Upregulation of Xcl1, E-Cadherin and TGFβ-Related Signaling in the Injury Microenvironment as Potential Mediators of CD103+ Cell Recruitment

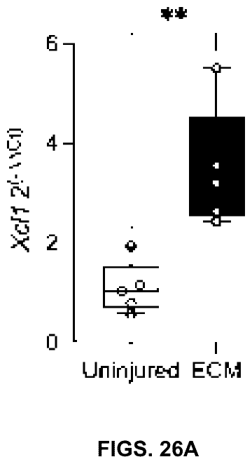

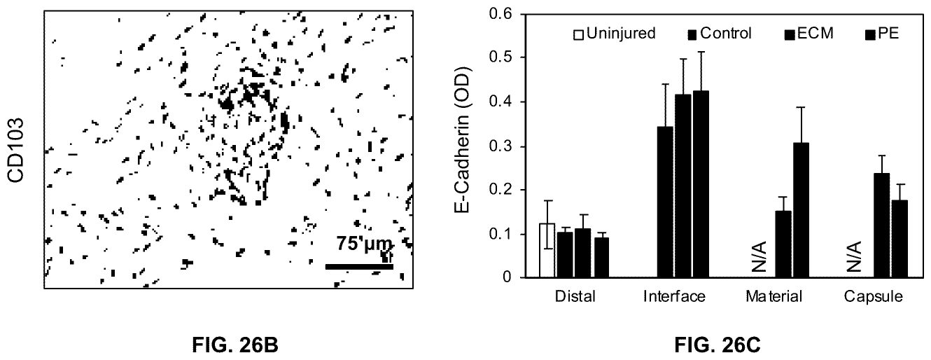

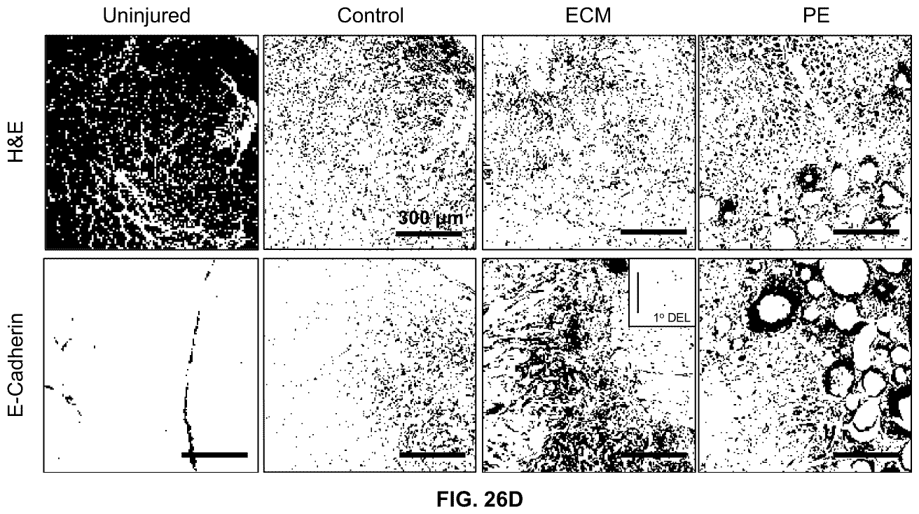

At 7 days post-injury, there was a significantly higher expression of Xcl1 mRNA in ECM-treated muscle injury in comparison with an uninjured control. (FIG. 6A). Within the muscle area, CD103+ cells were readily apparent in ECM treated injury with a variety of morphologies associated with both myeloid and lymphoid cells. These cells were located both at the injury interface, within the implant, and around the capsule (FIG. 26B). In certain cases, these cells were found within clusters of immune infiltration, but also dispersed throughout the injury site. Additionally, there was an up-regulation of E-Cadherin, the ligand for the integrin CD103, at the injury interface in comparison to more distal uninjured muscle tissue (FIG. 26C). This was true for all treatment groups, though most significant with biomaterial implantation—both ECM and PE induced this and was highly prevalent in damaged muscle tissue as well as in hematoxylin-dense areas of immune infiltrate (FIG. 26D).

When evaluating the cytokine and chemokine environment via protein arrays, there were large differences in the immune profile depending upon the presence of an injury along with treatment type. Material implantation correlated with an increase in CCL6, which is produced by neutrophils and macrophages that are enriched by these materials. Both PE and ECM treatment induced myeloperoxidase (MPO) upregulation associated with generation of reactive oxygen species (ROS). Interestingly, injury up-regulated Endoglin, a part of the TGFβ receptor, in comparison to an uninjured control, and TGFβ is a known inducer of CD103 expression (FIG. 27).

### Example 3. Examination of NK Cells Involved in Material Response

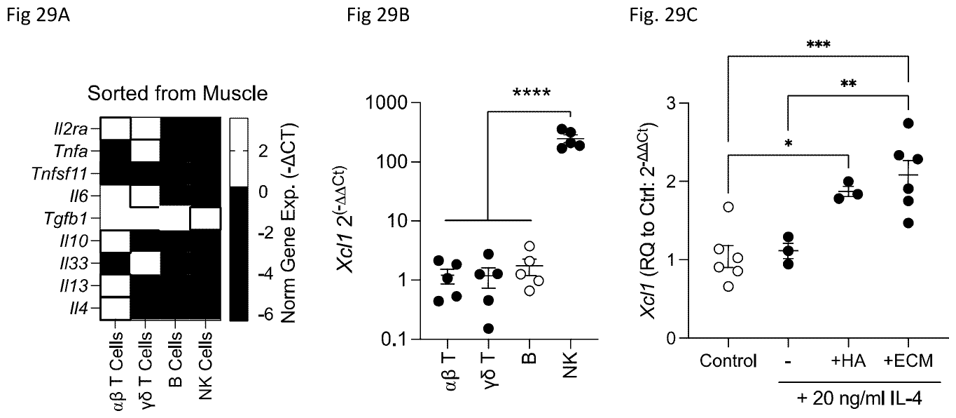

To further evaluate the immune response to biomaterials in injury, a 3 mm defect in the quadriceps muscle of mice was and three conditions investigated: untreated (control), polyethylene (PE) was implanted into the injury site inducing a profibrotic response, or extracellular matrix (ECM) was used to promote a pro-regenerative environment. The immune response at each site of trauma was then evaluated using flow cytometry, ELISA, and RT-PCR. At 7 days post injury, NK cells (CD49b⁺TCRβ⁻) were increased in ECM environment compared to PE and control (2.498% versus 0.216% and 0.0734%±0.017, P<0.0001). Gene expression analysis showed that theses NK cells displayed high levels of Tgfb1 expression (FIG. 29A). These NK cells were accompanied by an enrichment of XCR1⁺CD103⁺ conventional dendritic cells (cDC1s) that are capable of antigen cross-presentation. Furthermore, ECM treatment induced heightened levels of XCL-1 in the injury microenvironment and peripheral blood (FIG. 29B). There was no significant difference in the concentration of XCL-1 between wild type and Rag-1^{-/-} mice suggesting XCL-1 secretion is mediated by a RAG1-independent cell type such as NK cells. This correlated with an increase in XCR1⁺CD103⁺dendritic cells that promote regenerative behavior. In the absence of these cells in Batgf3^{-/-} mice, there were physical manifestations such as necrotic muscle fibers and giant cells more distal from the injury site showing a spread of trauma beyond the initial injury.

NK cells were also contacted, in vitro, with fragments of decellularized extracellular matrix (ECM) or low molecular weight hyaluronic acid (LMW-HA) and the level of Xcl1 production measured. At 24-hours post-exposure, up regulation of Xcl1 was observed, suggesting engagement of the NK cells with damage-associated molecular patters mediates Xcl1 secretion

Evaluating these data together, within the first week after injury there was in upregulation of XCL-1, in addition XCR1⁺CD103⁺dendritic cells and CD49b+NK Cells. The presence of XCL-1 provides a possible mechanism of XCR1⁺CD103+ cell recruitment. In Rag1^{-/-} mice, the concentration of XCL-1 was not significantly different compared to the wild type, indicating that a RAG-independent recruitment was the source of XCL-1. NK cells were found to be present at ECM injury site at a greater proportion when compared to the control and PE environment.

### Discussion of Results

The results demonstrate the induction of cross-presenting capable DCs by pro-regenerative materials in trauma. This is accompanied by MHCII-bearing M2 macrophages and by CD8+ iTregs and ST2+ regulatory B Cells in the periphery, as well as CD103+XCR1+ adaptive immune cells that are induced by trauma. Recruitment of cross-presenting capable dendritic cells and activation of CD103+XCR1⁺CD8 T cells peaked early during the response to injury and are at their maximum by 7 days post-injury. Up-regulation of both Xcl1 gene expression in homogenized injured tissue, and E-cadherin within the muscle injury site was detected, specifically at the interface of injury and material and in the capsule surrounding the material, which may serve as a mechanism for the recruitment of these CD103+ cells which could be binding the upregulated E-Cadherin for migration into the injury site. Furthermore, as Rag1^{-/-} mice had lower levels of CD103 expression on dendritic cells, and TGFβ secreted by T_{regs} is known to induce CD103 upregulation, this presents a possible mechanism of CD103 upregulation in DCs by injury associated TGFβ secretion by adaptive immune cells.

These data indicate a balance of antigen cross-presentation during recovery from wounding that is modulated by engineered material implantation. In the context of materials that promote type-2 and regulatory immune responses such as decellularized ECM scaffolds, this cross-presentation occurs in an environment that is more amendable to peripheral tolerance and antigen-specific wound repair. In the context of pro-inflammatory and pro-fibrotic materials such as PE, which favor a type-1 and type-17 immune response, this could lead to autoimmune activation and formation of auto-reactive T cells and antibodies leading to distal pathologies and systemic immune dysregulation that has been reported in some patients.

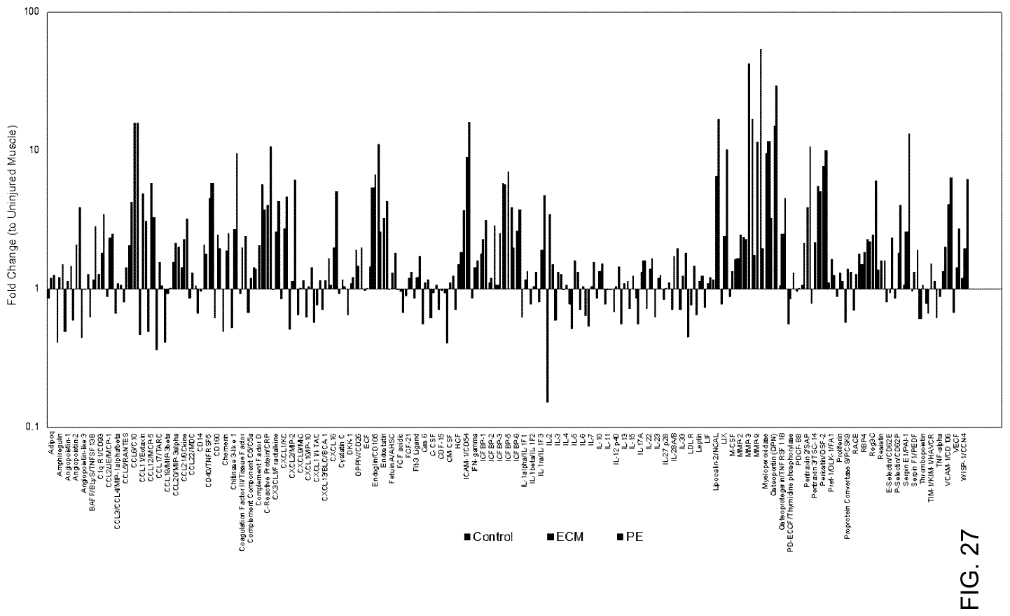

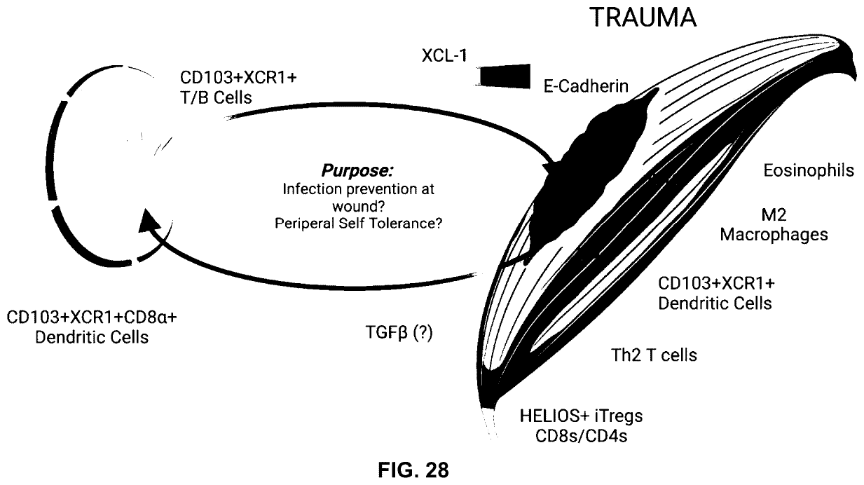

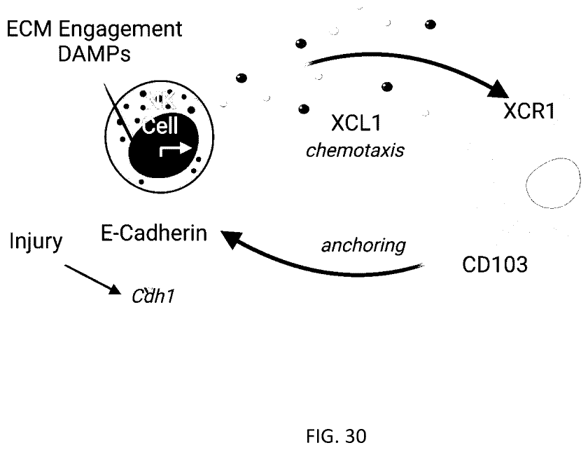

Presence of CD103+XCR1+ innate and adaptive immune cells may present a homeostatic regulation of response to injured self that are expanded during trauma after reaction with cross-presenting capable dendritic cells (FIGS. 28 and 30). Previous work has shown that XCR1+ T cells can be induced through trogocytosis and communication with cross-presenting capable dendritic cells and are a potential target for cancer immunotherapy. While the ECM scaffold introduces a protein source for new exogenous antigens, both the control injury as well as the PE-treated injury are surgically induced sterile trauma that does not introduce non-self-antigen, and thus, these cells are likely reacting to self-antigen, or in an antigen-independent manner. The cells and pathway in communication with cross-presenting capable dendritic cells have not been previously described in the context of trauma and biomaterial implantation and describe a novel mechanism of immune response to wounding and damaged self in traumatic injury.

1. A therapeutic composition for use in treating a wound of an individual, the therapeutic composition comprising a therapeutic agent that induces a pro-regenerative environment within the wound and/or within the tissue surrounding the wound wherein the therapeutic agent comprises decellularized extracellular matrix (ECM), or a component derived therefrom.

2. The therapeutic composition of claim 1, wherein the component derived therefrom comprises a degradation product of ECM.

3. The therapeutic composition of claim 1, wherein the therapeutic agent comprises one or more components selected from the group consisting of collagen, laminin, fibronectin, elastin, chondroitin sulfate, heparan sulfate, keratan sulfate, hyaluronic acid, perlecan, agrin, and degradation products thereof.

4. The therapeutic composition of claim 1, wherein the therapeutic agent comprises a matrikine or a damage associated molecular pattern (DAMP).

5. The therapeutic composition of claim 4, wherein the matrikine is selected from the group consisting of metastatin, affesten, canstatin, tetrastatin, pentastatin, lamstatin, hexastatin, endotrophin, restini, restin2, restin3, restin4, endostatin, neostatin, anastellin, sibsttin, PEX, endorepellin, CUB1CUB2 domain, Ten/2, Teni1/12/13, Ten14, kappa-elastin, ectodomain of syndecan-1, ectodomain of syndecan-2, ectodomain of syndecan-3, ectodomain of syndecan-4, elastokine, laminin peptide A13, laminin peptide C16, laminin 332 (laminin 5), a DGGRYY peptide, a GHK tripeptide, a VGVAPG peptide, a PGP tripeptide, an acetylated PGP tripeptide (AcPGP), tenascin-C (TNC), the G3 domain of nidogen-1, and tumstatin.

6. The therapeutic composition of claim 1, wherein the therapeutic agent induces local proliferation of M2 macrophages within the wound and/or within the tissue surrounding the wound.

7. The therapeutic composition of claim 1, wherein the therapeutic agent induces local proliferation and/or recruitment of conventional dendritic cells (cDC1s) within the wound and/or within the tissue surrounding the wound.

8. The therapeutic composition of claim 7, wherein the cDC1s are cross-presenting dendritic cells.

9. The therapeutic composition of claim 7, wherein the cDC1s are XCR1+CD103+ dendritic cells.

10. A medical device comprising the therapeutic composition of claim 1.

11. A kit comprising the therapeutic composition of claim 1.

12. A method of treating a wound in an individual comprising administering the therapeutic composition of claim 1 to the wound.

13. A method comprising administering to a wound in an individual, a composition that induces a pro-regenerative environment within the wound and/or within the tissue surrounding the wound, wherein the composition comprises decellularized extracellular matrix (ECM) or a component derived therefrom.

14. The method of claim 13, wherein the composition directs the immune response away from a Th1-type response within the wound and/or within the tissue surrounding the wound.

15. The method of claim 13, wherein the composition induces a Th2-type response within the wound and/or within the tissue surrounding the wound.

16. The method of claim 13, wherein the composition increases the number of M2 macrophages within the wound and/or within the tissue surrounding the wound.

17. The method of claim 13, wherein the composition induces an increase in the number of conventional dendritic cells (cDC1s) within the wound and/or within the tissue surrounding the wound.

18. The method of claim 17, wherein the cDC1s are cross presenting cDC1s or XCR+CD103+ dendritic cells.

19. The method of claim 13, wherein the component derived therefrom is a degradation product of ECM.

20. The method of claim 13, wherein the composition comprises:
a. one or more components selected from the group consisting of collagen, laminin, fibronectin, elastin, chondroitin sulfate, heparan sulfate, keratan sulfate, hyaluronic acid, perlecan, agrin, and degradation products thereof; or,
b. a matrikine.
